(19) United States
(12) Patent Application Publication
(10) Pub. No.: US 2025/0334596 A1
(43) Pub. Date: Oct. 30, 2025

(54) ISOTOPIC LABELS FOR QUANTITATIVE MASS SPECTROMETRY OF LIPID ISOMERS

(71) Applicant: THE TEXAS A&M UNIVERSITY SYSTEM, College Station, TX (US)

(72) Inventors: Xin Yan, College Station, TX (US); Hongyuan Yang, College Station, TX (US); Jiaxin Feng, College Station, TX (US); Shuli Tang, White Plains, NY (US); Gopal Reddy Ramidi, Bryan, TX (US); Syuan-Ting Kuo, College Station, TX (US)

(21) Appl. No.: 19/188,340

(22) Filed: Apr. 24, 2025

Related U.S. Application Data
(60) Provisional application No. 63/638,030, filed on Apr. 24, 2024.

(51) Int. Cl. G01N 33/92; G01N 33/58

(52) U.S. Cl. CPC G01N 33/92; G01N 33/58; G01N 2560/00

(57) ABSTRACT

The present disclosure provides compositions, kits, and methods for identifying one or more lipid isomers in a sample. By contacting the sample with one or more isotopic/isobaric labels to identify the one or more lipid isomers, the present disclosure provides means for quantification, concentration determination, identification of one or more double bond positions, geometry, and sn-position, and determination of a molar ratio of the lipid isomers

### CROSS-REFERENCE TO RELATED APPLICATIONS

This application claims the benefit under 35 U.S.C. § 119(e) of U.S. Provisional Application Ser. No. 63/638,030, filed on Apr. 24, 2024, the entire disclosures of which is incorporated herein by reference.

### GOVERNMENT RIGHTS

This invention was made with government support under CHE 2145487 awarded by NSF. The government has certain rights in the invention. Further, this invention was funded in part by a grant from The Welch Foundation under Welch grant number A-2089.

### GRANT INFORMATION

This invention was funded in part by a grant from The Welch Foundation under Welch grant number A-2089.

### BACKGROUND AND SUMMARY

Lipid metabolism plays functional roles in the dynamic regulation of cellular homeostasis, and eukaryotic cells use ˜5% of their genes and invest substantial resources in synthesising tremendous different lipids to fulfil the demand. Although progress has been made towards understanding the biochemical mechanism underlying lipid metabolism as well as the contributions of lipids to the disease process, precise structure characterization and quantitation of each individual lipid species at the isomer level are still elusive.

Lipids exhibit remarkable structural diversity, including a variety of isomers arising from different subclass, fatty acyl chain lengths, carbon-carbon double-bond positions and configurations. Lipids isomers, including the numbers and positions of carbon-carbon double bonds (C═C bonds), plays important roles in lipid metabolism, owing to the impressive biological functions of the structurally diverse lipids thus obtained. Since the conventional MS-based lipidomics often contain structural ambiguities or lack of clear structural evidence in identifying these modifications, lipid structure elucidation usually relies on biological intelligence or assumptions and thus may lead to misinterpretations. For example, many fatty acids with unusual sites of unsaturation have not been described by canonical pathways in prostate cancer cell lines until the application of isomer-resolved MS imaging technique. Moreover, in recent years, the changes in relative concentrations of lipid C═C positional isomers were recognized in the onset/progression of breast cancer.

Recently, several advanced strategies have been developed to enhance MS capabilities for resolving detailed lipid structures. Technical developments in analytical methods that aim at identification of lipid double bond position isomers include (i) hyphenation of additional separation instruments prior to MS analysis such as high-performance liquid chromatography (HPLC) and ion mobility spectrometer (IMS); (ii) assembling of novel gas-phase ion activation techniques, such as OzID, UVPD and EIEIO; and (iii) application of various chemical derivatization methods such as ozonolysis, Paternò-Büchi (PB) reaction and epoxidation, which enable the recognition of existing lipid C═C positional isomers hidden from conventional lipidomics. Additionally, identifying the geometric configuration (cis/trans) of lipid double bonds presents significant challenges. Current strategies mainly rely on separation of cis/trans isomers by liquid chromatography (LC) and IM-MS, with identification typically achieved by comparing elution or ion mobility arrival times to reference standards To distinguish sn-isomers in glycerophospholipids, methods including anion/cation addiction of lipids, radical-induced dissociation of bicarbonate-adducted lipids, CID coupled ozone-induced fragmentation, and coupling MS with ion mobility have been explored. Despite these technological advances, there still remains a need to accurately determine the quantity of each individual lipid isomers. In particular, the lack of proper internal standards (ISs) and calibration curves prohibit further advances in view of current methods.

Accordingly, the present disclosure provides novel methods utilizing an isotope tagging strategy to improve identification and quantification of lipids, as well as compositions and kits comprising isotopic/isobaric labels. The disclosure provides methods for identification and quantification of lipid isomers including C═C bonds positional isomer, geometric configuration (cis/trans) of lipid double bonds, and sn-positional isomers. Compared with the currently available methodologies, the disclosed methods include several advantages, including: (i) identification of lipid C═C positional isomers unambiguously and (ii) quantification of the concentration ratios of each lipid isomer species among multiple samples simultaneously in one experimental run; (iii) efficient conversion of lipid C═C bonds to aziridine products regardless of lipid categories and unsaturation degrees; (iv) improvement of ionization efficiencies of nonpolar lipids; and (v) excellent compatibility with various MS or LCMS platforms. These features along with a low sample consumption and simplified tagging procedure can enable its widespread applications in lipidomics.

### BRIEF DESCRIPTIONS OF THE DRAWINGS

The detailed description particularly refers to the accompanying figures in which:

FIGS. 1A-1C show the design of aziridination-based 2-aminopyridine isotopic (AAPI) tags for lipid analysis. FIG. 1A shows a schematic of 2-AP based aziridination reaction of lipid C═C bonds and characteristic fragments generated from CID-MS/MS of the lipid aziridines. FIG. 1B shows a schematic of duplex derivatization and simultaneous analysis of two samples using AAPI tags (2-AP-[d0]/2-AP-[d4]). FIG. 1C shows schematic of AAPI tags for identification of C═C positional isomers in two lipid samples unambiguously and quantification of the molar ratios simultaneously via tandem MS analysis.

FIGS. 2A-2F depict identification and quantification of FAEE lipid C═C positional isomers. FIG. 2A shows a chemical structure of FAEE 18:1 (Δ9), FAEE 18:1 (Δ11), the 2-AP derivatized FAEE 18:1 aziridine and their C═C position diagnostic ions upon CID-MS/MS. FIG. 2B shows a schematic of derivatization of FAEE 18:1 isomer mixtures using AAPI tags to validate its identification and quantification capabilities in lipidomics. FIG. 2C shows tandem mass spectra of FAEE 18:1-2-AP-[d0] and [d4] aziridine products derived from six samples and one control. FIG. 2D shows a calibration curve of FAEE 18:1 (Δ9)-2-AP-[d0] using FAEE 18:1 (Δ11)-2-AP-[d0] as IS. FIG. 2E shows a calibration curve of FAEE 18:1 (Δ9)-2-AP-[d0] using FAEE 18:1 (Δ9)-2-AP-[d4] as IS. FIG. 2F shows a calibration curve of FAEE 18:1 (Δ11)-2-AP-[d0] using FAEE 18:1 (Δ11)-2-AP-[d4] as IS.

FIG. 3 illustrates identification and quantification of lipid in different categories. Calibration curves achieved using lipid standards of different categories. Chemical structure of FA 18:1 (Δ9), PC 18:1 (Δ9)-18:1 (Δ9), TAG 18:1 (Δ9)-18:1 (Δ9)-18:1 (Δ9), CE 18:1 (Δ9) and their corresponding C═C bond diagnostics ions after 2-AP aziridination.

FIG. 4 shows the identification and quantification of FA 18:3 lipid. Schematic of 2-AP based aziridination reaction of lipid C═C bonds and characteristic fragments generated from CID-MS/MS of the lipid aziridines. Schematic of duplex derivatization and simultaneous analysis of two samples using AAPI tags (2-AP-[d0]/2-AP-[d4]). Schematic of AAPI tags for identification of C═C positional isomers in two lipid samples unambiguously and quantification of the molar ratios simultaneously via tandem MS analysis.

FIGS. 5A-5B show the one N-aryl aziridination-based ¹³C isotopic labeling strategy for isomer-level lipid structure characterization and relative quantification. FIG. 5A is a schematic depiction of the aziridination reaction, showing the introduction of a cleavable aziridine ring at the site of unsaturation. FIG. 5B shows an overview of the labeling workflow, in which separate samples are derivatized with either a “light” (¹²C) or “heavy” (¹³C) reagent before being combined for mass spectrometric analysis. The resulting mass difference allows direct relative quantification of lipid isomers without the need for calibration curves or internal standards.

FIGS. 6A-6G depict N-aryl aziridination enables comprehensive isomer-level lipid structure characterization. FIG. 6A shows a schematic representation of the aziridination reaction and subsequent CID fragmentation for PC 16:0/18:1 (9Z). Red numbers indicate DIs that pinpoint C═C locations and blue numbers highlight additional fragments. FIG. 6B shows a MS¹ spectrum of PC 16:0/18:1 (9Z) after aziridination and FIG. 6C shows a MS² spectrum showing diagnostic ions that reveal the C═C position. FIG. 6D shows a fragmentation pathway of TAG 18:1(9Z)/18:1(9Z)/18:1(9Z) after aziridination and FIG. 6E shows its MS² spectrum that reveals the C═C position. FIG. 6F shows a fragmentation pathway of FA 18:3 (9Z, 12Z, 15Z) (main product) after aziridination and FIG. 6G shows its MS² spectrum illustrating the dominant tagging positions. See FIG. 16A-16F for additional details on lipid characterization.

FIGS. 7A-7M show ¹³C isotopic labeling strategy for relative quantification of lipid isomers. FIG. 7A shows a structure of deuterium labeled 2-aminopyridine and ¹³C labeled 4-nitroaniline reagents used in this study. FIG. 7B shows extracted ion chromatograms (EICs) for TAG 18:1(9Z)/18:1(9Z)/18:1(9Z) and PC 18:1(9Z)/18:1(9Z) labeled with ¹³C reagent, demonstrating their co-elution while FIG. 7C shows corresponding EICs for deuterium-labeled lipids showing earlier elution compared to hydrogen-labeled counterparts. Red traces represent light-labeled species, and blue traces represent heavy-labeled species. FIG. 7D shows measured MS1 intensity ratios of ¹²C-labeled to ¹³C-labeled product peaks, and FIG. 7E shows D-labeled to H-labeled peaks. FIG. 7F shows a structure of the FAEE 18:1 after aziridination and the DIs of C═C at Δ9 and Δ11 location upon CID. FIG. 7G shows a schematic depicting FAEE 18:1 isomer mixtures derivatized with a ¹³C mass tag to enable relative quantification. FIG. 7H and FIG. 7I show MS² spectra of the light- and heavy-labeled products, respectively. FIG. 7J and FIG. 7K show linear correlations between DI intensity ratios and the initial concentration ratios for both the Δ9 and Δ11 isomers (n=3). FIG. 7L shows measured DI intensity ratios comparing light- and heavy-labeled lipids (n=3), and FIG. 7M shows the dynamic range of the ¹³C-labeled mass tag across multiple lipid classes with their limit concentration required for aziridination (n=3).

FIGS. 8A-8E show N-aryl aziridination-based ¹³C mass tagging for qualitative and quantitative lipidomic analysis of bovine heart and yeast extracts at the isomer level. FIG. 8A shows a workflow of the Python-based, semi-automated paired aziridine-lipid search. FIG. 8B shows an overview of identified and relatively quantified lipid C═C positional isomers in bovine heart and yeast extracts. FIG. 8C shows a plot of light-tagged versus heavy-tagged retention times (R²=1.000) demonstrating co-elution of ¹³C-labeled aziridine-lipids under RP-HPLC conditions in biological samples. FIG. 8D shows a measured DI intensity ratios (light vs. heavy) for yeast extracts, confirming a 2:1 concentration ratio (n=3). The dashed red line marks the theoretical DI ratio for each lipid. FIG. 8E shows a summary of results from bovine heart (n=44) and yeast (n=27), showing DI ratios consistently near 2 and underscoring the accuracy of this ¹³C-based quantification strategy.

FIGS. 9A-9C illustrate the total FA analysis in A 549 and MDA-MB-231 cells under SCD1 and FADS2 modulation. FIG. 9A shows sensitivity profiles of A 549 and MDA-MB-231 cells treated with CAY 10566 (SCD1 inhibitor) and SC-26196 (FADS2 inhibitor), normalized to DMSO control (n=3, unpaired two-sided Student's t-test). FIG. 9B shows a Log 2(fold change) of FA levels following SCD1 inhibition or dual (SCD1+FADS2) inhibition relative to control (n=3). FIG. 9C shows a heat map showing changes in total FA abundance for each treatment in both cell lines. Hierarchical clustering (Pearson correlation, complete linkage) groups samples by cell type and treatment condition.

FIGS. 10A-10G depict isomer-level FA profiling reveals differential FADS2 usage in A 549 versus MDA-MB-231 cells. FIG. 10A shows PCA using isomer-level FA data separates the two cell lines, whereas FIG. 10B shows sum-level FA profiles do not. FIG. 10C shows PCA highlighting that FADS2-derived FAs drive major differences between A 549 and MDA-MB-231. FIG. 10D shows examples of SCD1-desaturated FAs decrease after SCD1 inhibition in both cell lines. FIG. 10E shows FADS2-desaturated products increase only in A 549, then decline with FADS2 inhibition. FIG. 10F shows that some FAs show higher abundance of FADS1-derived isomers in A 549. FIG. 10G shows a metabolic pathway diagram illustrating the FA isomers identified, along with their desaturation and elongation routes.

FIGS. 11A-11D show the design and concept of tandem balance loss tag (TBLT) FIG. 11A shows the chemical structure of TBLT. FIG. 11B shows a tandem spectrum of the 4-plex TBLT. FIG. 11C shows a table of the alternative R group in mass balance part. FIG. 11D shows a synthetic route for non-isotopic tag.

FIGS. 12A-12I show the TBLT labeling and aziridination of lipids, where FIG. 12A shows aziridination and TBLT labeling of lipids. FIG. 12B, shows the general experiment workflow of 4-plex TBLT tag for relative quantification. FIG. 12C shows tandem spectrum of 4-plex tag labeled ethyl oleate. FIG. 12D shows the linear correlation between the measured ratios of reporter ion intensities and the concentration ratios of tag-labeled ethyl oleate. FIG. 12E shows the mass spectrum of 3 isomeric overlapped lipids: PE 18:0/18:1, PC15:0/18:1 and PCO 16:0/18:1. FIG. 12F shows the mass spectrum of tag labeled PE 18:0/18:1, PC 15:0/18:1 and PCO 16:0/18:1. FIG. 12G shows the reporter ion ratio of PC 15:0/18:1 and PCO 16:0/18:1 labeled by TBLT and TMT respectively. FIG. 12H shows the tandem MS of tag labeled PC15:0/18:1 and PCO 16:0/18:1 at m/z 1018.6 with the isolation window of 0.7 Th. FIG. 12I shows the tandem MS of tag labeled PE 18:0/18:1 at m/z 1275.8 with the isolation window of 0.7 Th.

FIGS. 13A-13J show characterization and quantification of lipids at multiple isomer levels. FIG. 13A shows the fragment pathway of FA 18:1 (n-9). FIG. 13B shows the tandem MS spectrum of mixture of 4 isobaric TBLT labeled FA 18:1 (n-7) and (n-9). FIG. 13C shows the preferred fragment pathway of TBLT labeled cis-FAEE and trans trans-FAEE. FIG. 13D shows an extracted ion chromatogram of TBLT labeled cis-FAEE and trans trans-FAEE. FIG. 13E shows a tandem MS spectrum of TBLT labeled cis-FAEE. FIG. 13F shows a tandem MS spectrum of TBLT labeled trans-FAEE. FIG. 13G shows a preferred fragment pathway of TBLT labeled PC 18:1/16:0 and PC 16:0/18:1. FIG. 13H shows an extracted ion chromatogram of TBLT labeled PC 18:1/16:0 and PC 16:0/18:1. FIG. 13I shows a tandem MS spectrum of TBLT labeled PC 18:1/16:0. FIG. 13J shows a tandem MS spectrum of TBLT labeled PC 16:0/18:1.

FIGS. 14A-14C show comprehensive structure analysis of phosphatidylcholine lipids. FIG. 14A shows the hierarchy of GP identification and characterization using the identification of PC 16:0/18:1(9Z) at each level as an example. FIG. 14B shows the chemical structure of tag labeled PC 16:0/18:1(9Z) and cleavage site to produce the diagnostic ions for isomer identification upon HCD. FIG. 14C shows the tandem spectrum of tag labeled PC 16:0/18:1(9Z).

FIG. 15 shows the workflow of deep profiling of lipid biological samples using TBLT labeling.

FIGS. 16A-16E show nano-ESI of a series of lipid standards (Table 1) to evaluate the labeling efficiency of N-aryl aziridination. FIG. 16A shows PC, FIG. 16B triacylglycerides (TAGs), FIG. 16C depicts CE18:1(9Z), and lipids with multiple C═C bonds on a single fatty acyl chain, FA 18:3 is shown in FIG. 16D. C═C DIs were also detected for PC 18:1(9Z)/18:1(9Z), CE 18:1(9Z), FA 18:1(9Z), and FAEE 18:1(9Z). FIG. 16E shows low-intensity DIs for the Δ12 position (m/z 231 and 345) were also observed.

FIGS. 17A-17F show the MS¹ spectra. The intensity ratios of diagnostic ions was monitored over the expected ratios.

FIGS. 18A-18F depict the calibration curves generated for the indicated lipid.

FIGS. 19A-19J show the mass fragmentation of TAG (FIG. 19A-FIG. 19B), PC (FIG. 19C-FIG. 19D), FA (FIG. 19G-FIG. 19H), FAEE (FIG. 19I-19J.

FIGS. 20A-20D shows the mass fragmentation patterns and the structures of the ions for TAG 54:1 (FIG. 20A), MG 18:1 (FIG. 20B), FA 18:1 (FIG. 20C), and PC 34:1 as shown in FIG. 20D.

FIGS. 21A-21E show the MS² of different lipids, where FIG. 21A shows the MS² chromatograph of light tab labeling of TAG 54:2. FIG. 21B shows the MS² chromatograph of heavy tab labeling of TAG 54:2. FIG. 21C shows the mass abundances of m/z 1159.86 (top) and m/z 1171.9 (bottom). FIG. 21D shows the mass abundances of 1159.86 (top) and 1171.9 (bottom). FIG. 21E shows similar mass abundances of 1159.86 (top) and 1171.9 (bottom).

FIG. 22 shows the diagnostic ion ratios for each of the lipid or lipid mixtures of the application.

FIGS. 23A-23C demonstrate that A 549 and MDA-MB-231 cancer cell lines were used as models to evaluate application of the aziridination-based ¹³C isotopic labeling mass tag and FA desaturase inhibitor. Stearoyl-CoA desaturase 1 (SCD1) and fatty acid desaturase 2 (FADS2) were selected as targets.

FIGS. 24A-24B show the composition of FA 16:1 isomer in A 549 cell after SCD inhibition, dual inhibition, and controls.

FIGS. 25A-25D show the structures of four isobaric tags.

FIG. 26 shows the detailed synthetic scheme of one of the isobaric tags (A).

FIG. 27 shows the detailed synthetic scheme of one of the isobaric tags (B).

FIG. 28 shows the detailed synthetic scheme of one of the isobaric tags (C).

FIG. 29 shows the detailed synthetic scheme of one of the isobaric tags (D).

FIG. 30 shows the correlation between measured ratios of mass reporters and expected concentration ratios, where ethyl oleate, oleic acid, PC 34:1 and PE 34:1 were tested.

### DETAILED DESCRIPTION

In an illustrative aspect, a method of identifying one or more lipid isomers in a sample is provided. The method comprises contacting the sample with one or more isotopic/isobaric labels to identify the one or more lipid isomers.

In an embodiment, the method comprises quantification of the one or more lipid isomers.

In an embodiment, the method comprises quantification of a concentration of the one or more lipid isomers. In an embodiment, the method comprises identification of one or more C═C double bond positions, their geometry (cis/trans), and sn-position of the one or more lipid isomers of the one or more lipid isomers.

In an embodiment, the method comprises identification of one or more double bond positions, their geometry (cis/trans), and sn-position of the one or more lipid isomers. In an embodiment, the method comprises identification of one or more C═C double bond positions, their geometry (cis/trans), and sn-position of the one or more lipid isomers. In an embodiment, the method comprises quantification of one or more C═C double bond positions, their geometry (cis/trans), and sn-position of the one or more lipid isomers.

In an embodiment, the method comprises determination of a molar ratio of the one or more lipid isomers.

In an embodiment, the method provides identification at an accuracy of greater than 90%.

In an embodiment, the one or more lipid isomers are selected from the group consisting of fatty acids (FA), glycerophospholipids (GPL), unsaturated fatty acid derivatives, cholesteryl ester (CE), triacylglycerides (TAG), yeast polar extracts, and any combination thereof. In an embodiment, the one or more lipid isomers comprise fatty acids (FA). In an embodiment, the one or more lipid isomers comprise glycerophospholipids (GPL). In an embodiment, the one or more lipid isomers comprise unsaturated fatty acid derivatives. In an embodiment, the one or more lipid isomers comprise cholesteryl ester (CE). In an embodiment, the one or more lipid isomers comprise triacylglycerides (TAG). In an embodiment, the one or more lipid isomers comprise yeast polar extracts.

In an embodiment, the method further comprises administering collision-induced dissociation (CID) to the sample. In an embodiment, the CID provides fragmentation of the lipid isomers, wherein the lipid isomers are labeled with the one or more isotopic labels. For instance, aziridination allows efficient conversion of lipid C═C bonds to the aziridine products, which generate diagnostic ions via the cleavage of the three-membered aziridine ring upon CID fragmentation, to identify original lipid C═C bond positions. The method can also comprise administering collision-induced dissociation (HCD) to the sample. In an embodiment, the HCD provides fragmentation of diagnostic ions coupled with mass reporter, enabling simultaneous identification and quantification of lipid sn-positional isomers, double bond positional isomers, and their geometric (cis/trans).

In an embodiment, the method further comprises administering liquid chromatography (LC) to the sample. In an embodiment, the method further comprises administering mass spectroscopy (MS) to the sample. In an embodiment, the method further comprises administering tandem mass spectroscopy (MS/MS) to the sample. In an embodiment, the method further comprises administering liquid chromatography-mass spectrometry (LC-MS) to the sample. In an embodiment, the method further comprises administering high-performance liquid chromatography-mass spectrometry (HPLC-MS) to the sample.

In an embodiment, the method does not comprise use of an internal standard to identify the one or more lipid isomers. In an embodiment, the method does not comprise use of a calibration curve to identify the one or more lipid isomers.

In an embodiment, the one or more isotopic labels comprises an aziridination-based labeling. In an embodiment, the one or more isotopic labels comprises a deuterium-based labeling. In an embodiment, the one or more isotopic labels comprises a 2-aminopyridine (2-AP)-based labeling.

In an embodiment, the one or more isotopic labels comprises a ¹³C-based labeling. In an embodiment, the one or more isotopic labels comprises a 4-nitroaniline-based labeling. In an embodiment, the one or more isotopic labels comprises an N-aryl-based labeling.

In an embodiment, the one or more isotopic labels comprises an aziridination-based and a 2-AP-based labeling. In an embodiment, the one or more isotopic labels comprises an aziridination-based and a deuterium-based labeling. In an embodiment, the one or more isotopic labels comprises a 4-nitroaniline-based N-aryl aziridination labeling. In an embodiment, the one or more isobaric labels comprises an aziridination-based and a tandem balance loss tag (TBLT) labeling.

In another illustrative aspect, a method of quantifying one or more lipid isomers in a sample is provided. The method comprises contacting the sample with one or more isotopic/isobaric labels to quantify the one or more lipid isomers. The previously described embodiments of the method of identifying one or more lipid isomers in a sample are applicable to the method of quantifying one or more lipid isomers in a sample as described herein.

In another illustrative aspect, a method of quantifying a concentration of the one or more lipid isomers in a sample is provided. The method comprises contacting the sample with one or more isotopic/isobaric labels to quantify the concentration of the one or more lipid isomers. The previously described embodiments of the method of identifying one or more lipid isomers in a sample are applicable to the method of quantifying a concentration of one or more lipid isomers in a sample as described herein.

In another illustrative aspect, a method of identifying one or more double bond positions of one or more lipid isomers in a sample is provided. The method comprises contacting the sample with one or more isotopic/isobaric labels to identify the one or more double bond positions of the one or more lipid isomers. The previously described embodiments of the method of identifying one or more lipid isomers in a sample are applicable to the method of one or more double bond positions of one or more lipid isomers in a sample as described herein.

In another illustrative aspect, a composition comprising one or more reagents is provided, wherein the reagents are selected from the group consisting of 4-nitroaniline, [¹³C₆]-4-nitroaniline, iodosobenzene (PhIO), Rh₂(esp)₂ catalyst, and any combination thereof.

In an embodiment, the composition further comprises a solvent. In an embodiment, the solvent is hexafluoroisopropanol (HFIP).

In an illustrative aspect, a kit comprising i) one or more reagents, wherein the reagents are selected from the group consisting of 4-nitroaniline, [¹³C₆]-4-nitroaniline, iodosobenzene (PhIO), Rh₂(esp)₂ catalyst, and any combination thereof and ii) a solvent is provided.

In an embodiment, the solvent is hexafluoroisopropanol (HFIP). In an embodiment, the kit further comprises dichloromethane (DCM). In an embodiment, the kit further comprises one or more microcentrifuge tubes. In an embodiment, the kit further comprises one or more glass vials. In an embodiment, the kit further comprises one or more reverse phase columns. In an embodiment, the kit further comprises instructions for use. In an embodiment, the kit further comprises a mass spectrometer (MS) with a liquid chromatography (LC) system. In an embodiment, the kit further comprises a mass spectrometer (MS) with a high-performance liquid chromatography (HPLC) system.

In another illustrative aspect, a composition comprising one or more reagents, wherein the reagents are selected from the group consisting of N-Boc-O-tosyl hydroxylamine, TsONHBoc TBLT-4plex reagent, 1M triethyl ammonium bicarbonate, and any combination thereof is provided.

In an embodiment, the composition further comprises a solvent. In an embodiment, the solvent is hexafluoroisopropanol (HFIP).

In an illustrative aspect, a kit comprising i) one or more reagents, wherein the reagents are selected from the group consisting of N-Boc-O-tosyl hydroxylamine, TsONHBoc TBLT-4plex reagent, 1M triethyl ammonium bicarbonate, and any combination thereof, and ii) a solvent is provided.

In an embodiment, the solvent is hexafluoroisopropanol (HFIP). In an embodiment, the kit further comprises anhydrous dimethylformamide. In an embodiment, the kit further comprises anhydrous ethanol. In an embodiment, the kit further comprises one or more lipid extraction solvents. In an embodiment, the kit further comprises ammonium bicarbonate. In an embodiment, the kit further comprises hydroxylamine. In an embodiment, the kit further comprises a C18 reversed-phase column. In an embodiment, the kit further comprises instructions for use. In an embodiment, the kit further comprises a mass spectrometer (MS) with a liquid chromatography (LC) system. In an embodiment, the kit further comprises a mass spectrometer (MS) with a high-performance liquid chromatography (HPLC) system.

### EXAMPLES

### Example 1

### Materials and Methods of Examples 1-6

### Materials and Reagents

The glycerophospholipid (GPL) standards and yeast polar lipid extract (S. cerevisiae) used in this work were purchased from Avanti Polar Lipids (AL, U.S.A.). Fatty acid (FA) 18:1 (Δ6), FA 18:1 (Δ9), FA 18:3 (Δ6, Δ9, Δ12), FA 18:3 (Δ9, Δ12, 1Δ5), FA 20:4 (Δ5, Δ8, Δ11, Δ14), fatty acid ethyl ester (FAEE) 18:1 (Δ9), FAEE 18:1 (Δ11), FAEE 18:2 (Δ9, Δ12), FAEE 20:4 (Δ5, Δ8, Δ11, Δ14), cholesterol ester (CE) 18:1 (Δ9), CE 18:1 (Δ11), CE 18:2 (Δ9, Δ12), CE 20:4 (Δ5, Δ8, Δ11, Δ14), triacylglyceride (TAG) 18:1 (Δ9), TAG 18:1 (Δ11), and TAG 18:2 (Δ9, Δ12) were purchased from Nu-Chek Prep, Inc. 2-Aminopyridine (2-AP-[d0]) was purchased from TCI Chemical, Inc. 2-Aminopyridine-[d6] (2-AP-[d6]) was purchased from C/D/N Isotopes Inc. Rh₂(es)₂ was purchased from Ambeed, Inc. Iodosobenzene (PhIO) was purchased from Aaron Chemical, Inc, Hexafluoro-2-propanol (HFIP) was from Chem-Impex Int'l. Inc. Acetonitrile (ACN), Isopropanol (IPA), water (H₂O), ammonium formate, and formic acid were from Sigma-Aldrich (St. Louis, MO, U.S.A.). All chemicals were used without purification.

### Aziridination of Unsaturated Lipids

The general procedure for aziridination of unsaturated lipids was using 5 mM 2-AP-[d0] or [d6], 3 mM PhIO and 0.5 mM Rh₂(esp)₂ in 1.0 mL HFIP solution while the lipid concentration varies from 80 nM to 1 mM. The resulting reaction solution was then stirred at room temperature for 2-24 hrs. The resulting solutions was then collected for MS analysis in the existence of 0.1% formic acid.

nESI-MS Analysis

The nESI tips were pulled from borosilicate glass capillaries (1.5 mm o.d. and 0.86 mm i.d., purchased from World Precision Instruments, Sarasota, FL, U.S.A.) using a P-1000 micropipette puller (Sutter Instrument, Novato, CA). All nESI-MS analysis was conducted using an Orbitrap Velos Pro Hybrid Ion Trap-Orbitrap mass spectrometer (Themo Fisher Scientific) for high-resolution MS and ion trap MS²/MS³ CID experiments. Samples were ionized in positive ion mode with spray voltages at 1.8 kV. S-lens RF level was set to 67.9%, and the capillary temperature was set at 200° C. Full MS scans were acquired at m/z 150-1200 with a resolving power of 60000 in Orbitrap FT mode. A maximum injection time of 500 ms and 1 microscan were used for full MS scans. MS²/MS³ experiments were performed using ion trap CID with the isolation width set at 1.5 Th. A maximum injection time of 500 ms and 2 microscans were used for tandem MS scans. CID energy used for fragmentation was around 30 arbitrary units. In re-ported mass spectra, the m/z values were rounded to four decimal places when the Orbitrap mass analyzer was used for data collection, while one decimal place was used for data collected from the ion trap mass analyzer.

### RPLC-MS Analysis

RPLC-MS analysis was conducted on a Vanquish HPLC system (Themo Fisher Scientific) hyphenated with an Orbitrap Velos Pro Hybrid Ion Trap-Orbitrap mass spectrometer (Themo Fisher Scientific) for high-resolution MS and ion trap MS²/MS³ CID experiments. Aliquots of 2 μL of un-derivatized samples or 2-AP derivatized samples were separated on a Accucore C30 column (Themo Fisher Scientific, 2.1 mm×150 mm, 2.6 μm). Mobile phase A in the chromato-graphic method included 60:40 water/ACN in 10 mM ammonium formate and 0.1% formic acid, and mobile phase B included 90:10 IPA/ACN, also with 10 mM ammonium formate and 0.1% formic acid. The LC pump was programmed at 0.2 mL/min flow rate. The optimal chromatographic gradient program was as follows: 30% B at 0-3 min, 30-43% B at 3-8 min, 43-50% B at 8-9 min, 50-90% B at 9-18 min, 90-99% B at 18-26 min, and 99% B at 26-30 min followed by 5 min re-equilibrium at 30% B. A heated electrospray ionization (HSEI) probe was equipped, whereas the spray voltage was set to 4.0 kV for positive ion mode and 3.2 kV for negative ion mode. The heated capillary and the HSEI probe were held at 250 and 350° C., respectively. The sheath gas flow was set to 35 psi, and the auxiliary gas was set to 15 psi.

### Example 2

### Design of Aziridination-Based 2-Aminopyridine Isotopic (AAPI) Tags

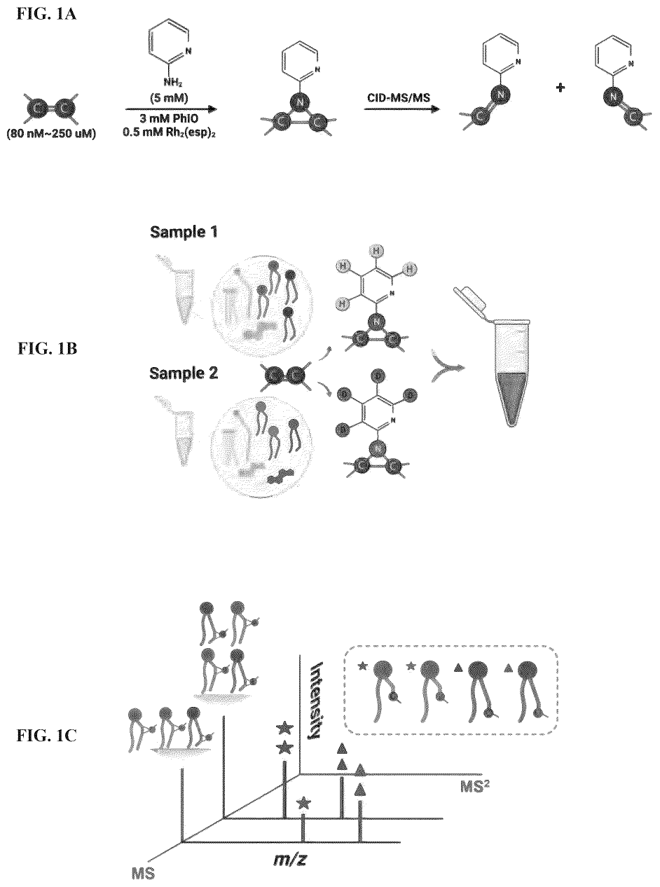

The design of aziridination-based 2-aminopyridine isotope (AAPI) tags aims at introducing distinct mass additions to lipids and thus the identical lipids derived from different samples have different masses. 2-AP based aziridination allows efficient conversion of lipid C═C bonds to the aziridine products, which generate diagnostic ions via the cleavage of the three-membered aziridine ring upon CID fragmentation, to pinpoint original lipid C═C bond positions (FIG. 1A). Meanwhile, the four H (¹H) atoms on the pyridine ring of the “light tag (2-AP-[d0])” are substitute with D (²H) to generate the “heavy tag (2-AP-[d4])”, enabling the duplex derivatization and simultaneous analysis of both the treatment and control group in one experimental run (FIG. 1B). Since the fragment information of lipids from different samples are collected in discrete MS² or MSⁿ spectra, their C═C bond positional isomer compositions could be identified and differentiated unambiguously. More importantly, determination of the molar ratios of those isomers could then be achieved by simply comparing the diagnostic ion intensities between the two MS² or MSⁿ spectra without the use of ISs or calibration curves (FIG. 1C). Compared to isobaric tags such as TMT, the developed AAPI tags fully exploit the potential of tandem MS for lipid structure characterization and quantification at isomer levels.

The advantage of 2-AP as the aziridination and isotopic labelling reagent is apparent considering the fact that the ionization efficiencies can be significantly improved via the introduction of N-pyridine group when comparing with previously reported N-tosyl (N-Ts) or N-Me groups, for MS analysis of nonpolar lipids, i.e., unsaturated fatty acid derivatives, cholesteryl ester (CE), triacylglycerides (TAG). Moreover, the high energies required for the cleavage of the strong N-pyridine bonds might make the alternate fragmentation pathways to generate more C═C bond position diagnostic ions dominant. In contrast, the N-carbonyl bond of the TMT labelled lipid aziridines are more liable to fracture.

### Example 3

### Identification and Quantification of FAEE C═C Positional Isomers

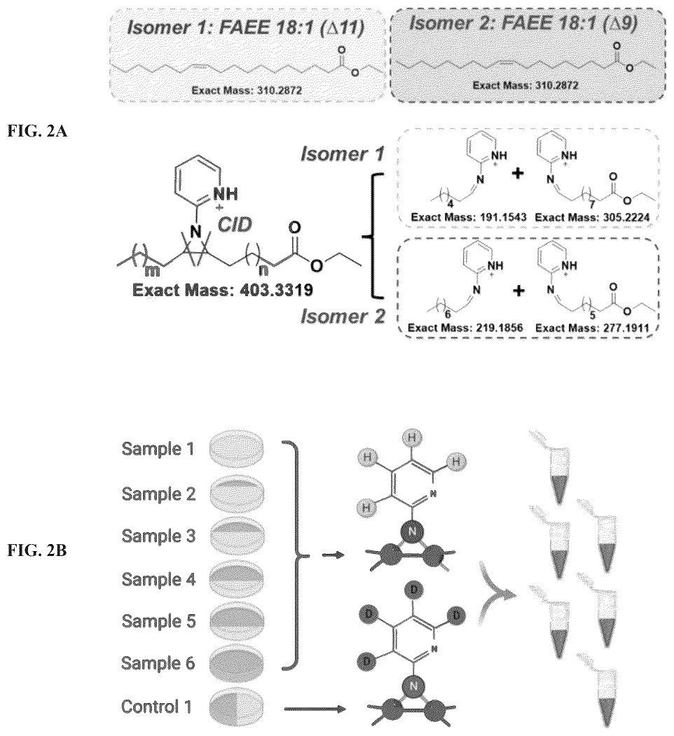

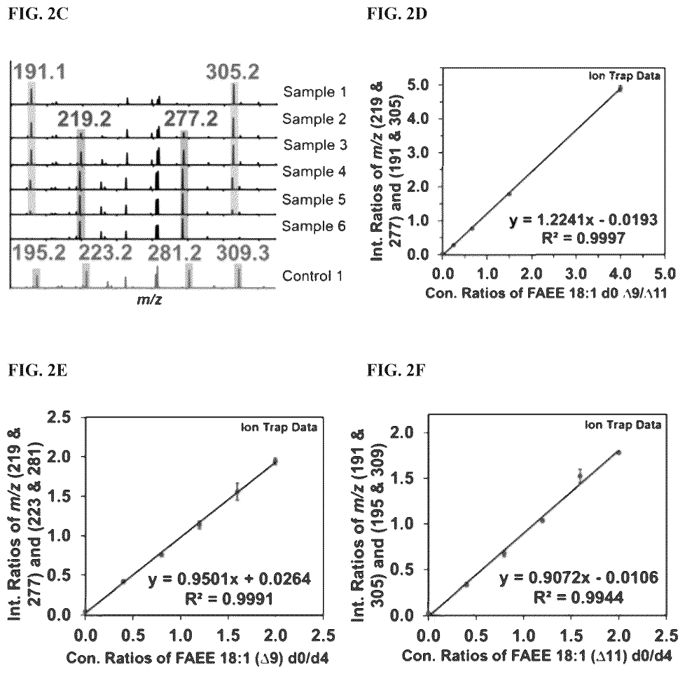

To validate the feasibility of the AAPI tags in the identification and quantification of lipid isomers, a series of lipid mixtures of fatty acid ethyl ester (FAEE) 18:1 (Δ9) and FAEE 18:1 (Δ11) was employed. It was anticipated that CID fragmentation of 2-AP-[d0] derivatized FAEE 18:1 (Δ11) could generate a pair of C═C diagnostic ions at m/z 191.2 and 305.2, while for 2-AP-[d0] derivatized FAEE 18:1 (Δ9), another pair of diagnostic ions at m/z 219.2 and 277.2 could be obtained (FIG. 2A). Similarly, fragments at m/z 195.2 & 309.3 and 223.2 & 281.2 are diagnostic ions for FAEE 18:1 (Δ11)-(2-AP)-[d4] and FAEE 18:1 (Δ9)-(2-AP)-[d4], respectively. During the experiments, six samples were labelled using the “light tag” 2-AP-[d0], with their total concentration of FAEE 18:1 kept constant at 100 μM, and the molar percentage (mol %) of FAEE 18:1 (Δ9) isomer varied from 0%, 20%, 40%, 60%, 80% to 100%. Meanwhile, a FAEE 18:1 mixture containing 50 μM FAEE 18:1 (Δ9) and 50 μM FAEE 18:1 (Δ11) was labelled with “heavy tag” 2-AP-[d4] and served as control. After AAPI labelling, the six 2-AP-[d0] derivatized samples were mixed with the 2-AP-[d4] derivatized control in 1:1 ratio by volume for later nESI-MS analysis with 0.1% formic acid (FIG. 2B). Two precursor ions at m/z 403.3313 and 407.3555 could then be found in the MS¹ spectra, which were the FAEE 18:1-2-AP-[d0] and [d4] products, respectively. CID of ions at m/z 403.3 generated two pairs of diagnostic ions, 191.2 & 305.2 for FAEE 18:1 (Δ11) and 219.2 & 277.2 for FAEE 18:1 (Δ9), with their intensities varied as the mol % of FAEE 18:1 (Δ9) was different among the six samples. In the meanwhile, CID of ions at m/z 407.4 derived from the control solution produces fragments at m/z 195.2 & 309.3 and 223.2 & 281.2, respectively (FIG. 2C).

In most conventional lipidomics, the total ion intensity ratios of the two pairs of C═C position diagnostic ions for (FAEE) 18:1 (Δ9) and FAEE 18:1 (Δ11) were then plotted against their corresponding molar ratios, and thus a calibration curve could be obtained to achieve quantification of the certain lipid isomers in unknown samples (FIG. 2D). As expected, a good linear relationship with R²=0.9997 was obtained with the slope as 1.2241. The results indicated that lipid FAEE 18:1 (Δ9) can produce more C═C bond positional diagnostic ions comparing to FAEE 18:1 (Δ11) under identical concentration and experimental conditions, and thus proving that the necessity of calibration curves to compensate for the distinct cleavage efficiencies at different C═C positions. However, since the developed AAPI tags allowed parallel tandem MS analysis of lipids from different samples simultaneously, the molar ratios of lipid isomers could then be determined by the intensity ratios of the isomer-specific diagnostic ions collected from two tandem spectra. The intensity ratios of fragments at m/z 219.2 & 277.2 for FAEE 18:1 (Δ9)-2-AP-[d0] and m/z 223.2 & 281.2 for FAEE 18:1 (Δ9)-2-AP-[d4] were then plotted against their molar ratios, and a good linearity with R²=0.9991 was obtained and the slope was 0.9501 with the y intercept as −0.0193 (FIG. 2E). As for FAEE 18:1 (Δ11), a good linearity (R²=0.9944) with a slope of 0.9072 and the y intercept of −0.0106 was achieved by plotting the diagnostic ion intensity ratios of m/z 191.2 & 305.2 and m/z 195.2 & 309.3 (FIG. 2F) against their molar ratios (FIG. 2F). These results indicated that diagnostic ion intensity ratios obtained after AAPI tagging could quantify the molar ratios of the lipid C═C positional isomers among different samples without the interference of other existing isomers.

To further validate the quantification capability of AAPI tags across a broader dynamic range, a series of FAEE 18:1 lipid solution was prepared at the concentration of 0.4 μM, 2 μM, 10 μM, 50 μM, 100 μM to 250 UM and derivatized using 2-AP-[d0], while a 50 μM FAEE 18:1 lipid solution was labelled by 2-AP-[d4]. Each of these included 50 mol % of FAEE 18:1 (Δ9) and 50 mol % FAEE 18:1 (Δ11). After AAPI tagging, the 2-AP-[d0] and 2-AP-[d4] derivatized lipid aziridines were mixed in 1:1 volume ratio for nESI-MS analysis with 0.1% formic acid. Two linear relationships were achieved with a slope of 1.0021 for lipid FAEE 18:1 (Δ9) (R²=0.9996) and 0.9031 for FAEE 18:1 (Δ9) (R²=0.9995) by plotting their diagnostic ions intensities ratios against the molar ratios, proving that quantification could be achieved with more than two orders of magnitude dynamic range. In addition, the precursor ions at m/z 403.3313 and 407.3555 can also be used to determine the molar ratios of FAEE 18:1 derived from different samples, although the C═C bond position information cannot be obtained until further fragmentation.

### Example 4

### Identification and Quantification of Lipids in Various Categories

Lipids are known to be fatty acids and their derivatives and substances related to biosynthetically or functionally to these compounds and can be classified into different categories owing to distinct chemical features such as fatty acids (FA), glycerophospholipids (GPL), triacylglycerides (TAG) and cholesterol esters (CE), etc. The structures of those lipids are composed of one or more fatty acids ester-linked to the glycerol or cholesterol backbones. Considering the abundant fatty acids in biological systems, the various combination of them gives rises to many different lipid species within the same categories. Therefore, the quantitative accuracy and general applicability of the designed AAPI tags should be premised on the basis that complete conversion of lipid C═C bonds and minimal side reactions were achieved regardless of lipid categories, the numbers or types of linked FA chains, and concentrations in a specific dynamic range. Therefore, a series of experiments were performed to optimize the reaction conditions of 2-AP based aziridination.

Phosphatidylcholine (PC) 18:1 (Δ9)-18:1 (Δ9) contains two identical monounsaturated fatty acyl chains and can easily be detected in MS analysis owing to the positively-chargeable phosphocholine group. Hence, PC 18:1 (Δ9)-18:1 (Δ9) was first employed to investigate the effects of reagents concentration on lipid aziridination and the conversion rate was roughly calculated by the MS intensity ratio of the dominant lipid aziridines and all lipid-related species. It was found that ˜96% of PC 18:1 (Δ9)-18:1 (Δ9) lipids could be converted to di-aziridine products with large excess of 2-AP reagent relative to lipid while the concentration of 2-AP was kept at least 5 mM. In this case, PC 18:1 (Δ9)-18:1 (Δ9) lipids at the concentrations of 10 μM, 100 μM and 250 μM could have ˜96% conversion rate within 2 hrs., and it took more than 18 hrs. to convert 1 mM PC lipids to ˜86% di-aziridines and ˜10% mono-aziridines. Meanwhile, a lower concentration of 2-AP reagent could lead to insufficient aziridination and competing side reactions. As for TAG lipids having three FA chains, i.e., TAG 18:1 (Δ9)-18:1 (Δ9)-18:1 (Δ9), the reaction time required to completely convert TAG to the tri-aziridine products increased to 6 hrs. when TAG lipid was 250 μM with 5 mM 2-AP reagent. CE 18:1 (Δ9) contains one C═C bond within its FA chain while the cholesterol backbone processes another C═C bond. 2-AP based aziridination of CE 18:1 (Δ9) allows the derivatization of both of the two C═C bonds within 2 hrs. when higher concentrated 2-AP reagent (15 mM) was used.

The feasibility of AAPI tags across a variety of lipid categories was then investigated under the optimized conditions using FA 18:1, PC 18:1-18:1, TAG 18:1-18:1-18:1 and CE 18:1 lipid standard. A series of lipid mixtures of FA 18:1 (Δ6) and FA 18:1 (Δ9) was prepared in the two protocols: i) the total concentration of FA 18:1 was kept constant at 100 μM with the mol % of FA 18:1 (Δ6) varied from 0%, 20%, 40%, 60%, 80% to 100% and (ii) FA 18:1 lipid solution was prepared at the concentration of 0.4 μM, 2 μM, 10 μM, 50 μM, 100 μM to 250 μM, which included 50 mol % of FA 18:1 (Δ6) and 50 mol % FA 18:1 (Δ9). These solutions were derivatized using 2-AP-[d0] at a concentration of 5 mM, while a solution containing 25 μM FA 18:1 (Δ6) and 25 μM FA 18:1 (Δ9) was labelled by 2-AP-[d4] (5 mM) as control. After AAPI tagging, the 2-AP-[d0] and 2-AP-[d4] derivatized lipid aziridines were mixed in 1:1 ratio for nESI-MS analysis with 0.1% formic acid. CID of 2-AP derivatized FA 18:1 (Δ6) and FA 18:1 (Δ9) could generate diagnostic ions to locate C═C bond positions. When the intensity ratios of diagnostic ions for FA 18:1 (Δ6)-2-AP-[d0] (m/z 207.1 & 261.2) and FA 18:1 (Δ6)-2-AP-[d4] (m/z 211.1 & 265.2) were plotted against their molar ratios, both of two linear relationships obtained had a slope very close to 1.00 and R-square values as 0.9982 and 1.000, respectively. Similar results for FA 18:1 (Δ9) (m/z 219.1 & 249.2 with 2-AP-[d0] and m/z 223.2 & 253.2 with 2-AP-[d4]) were also achieved either when the FA 18:1 was 100 μM with isomer mol % varied or when the FA 18:1 isomer mixtures were prepared across more than two orders of magnitude dynamic range. These results proved that the AAPI tags could achieve identification of lipid FA 18:1 C═C positional isomers unambiguously and quantification regardless of isomer mol % or the concentration of lipid within at least two orders of magnitudes.

While the C═C positional diagnostic ions for FA and FAEE lipids were obtained via CID-MS², multi-stage CID fragmentation was required to generate sufficient C═C position diagnostic ions for GPLs and CE lipids. In GPLs, two FAs and a phosphate are ester-linked to the glycerol backbone, while any one of several possible substituents is also linked to the phosphate moiety and therefore produced various kinds of GPLs including PC, PA, PG, PE, etc. CID of 2-AP derivatized PC 18:1-18:1 at m/z 970.7 would generate two major fragment ions at m/z 911.6 and 787.6, which corresponded to the neutral loss of trimethylamine (59 Da) and phosphocholine headgroup (183 Da). Therefore, further CID of the headgroup-loss product ions at m/z 787.6 was required to obtain the diagnostic ions for the identification of C═C bond positions. CID-MS³ of PC 18:1 (Δ9)-18:1 (Δ9)-2-AP-[d0] and [d8] (di-aziridines) yields diagnostic ions at m/z 569.4 and 571.4. While for PC 18:1 (Δ6)-18:1 (Δ6), the C═C positional diagnostic ions were obtained at m/z 527.4 and 531.4, respectively. In the quantitative analysis of lipid PC 16:0-18:1 (Δ9), PG 16:0-18:1 (Δ9), PA 16:0-18:1 (Δ9) and PE 16:0-18:1 (Δ9), CID-MS2 of their 2-AP derivatized products generated the dominant fragments that corresponded to the loss of phosphocholine, phosphoglycerol, phosphoric acid and phosphoethanolamine headgroup, respectively. And CID-MS³ of the headgroup-lost fragments could then produce the diagnostic ions for locating C═C bond positions in these GPLs. Since TAG lipids have FAs ester-linked to each of the three OH groups within the glycerol backbone, sufficient C═C positional diagnostic ions could be obtained in CID-MS² for identification and quantification purpose compared with GPLs. CID of TAG 18:1 (Δ9) 2-AP-[d0] and [d12] (tri-aziridines) generated diagnostic fragments at m/z 943.7 and 951.7, and for TAG 18:1 (Δ11), the diagnostic ions were obtained at m/z 971.8 and 979.8.

CE lipids contain one FA chain ester-linked to the cholesterol backbone. Similarly, CID of CE 18:1-2-AP-[d0] at m/z 835.7 would generate fragment ions at m/z 461.3 and 375.3, which corresponded to [Chol+2-AP+H—H₂O]⁺ and 2-AP labeled fatty acyl [FA 18:1+2-AP+H]⁺ ions. And followed CID-MS³ of 2-AP labeled fatty acyl ions was needed for locating CE lipid C═C bond positions. CID-MS³ of CE 18:1 (Δ9) 2-AP-[d0] and [d8] (di-aziridines) generated diagnostic fragments at m/z 219.2 & 249.2 and 223.2 & 253.2. While for CE 18:1 (Δ11), the diagnostic ions were obtained at m/z 191.2 & 277.2 and 195.2 & 281.2.

When the intensity ratios of diagnostic ions for PC 18:1-18:1, TAG 18:1-18:1-18:1 and CE 18:1 C═C positional isomers were plotted against their molar ratios, good linear relationships were obtained either when the lipid concentration was 100 μM with isomer mol % varied or when the lipid isomer mixtures were prepared across more than two orders of magnitude dynamic range. The slopes of these calibration curves were very close to 1.00 with R-square values above 0.995, which proved that the AAPI tags could achieve identification of lipid C═C positional isomers unambiguously and quantification of those isomers regardless of lipid categories, the numbers of linked FA chains, isomer mol % or the concentration of lipid for more than two orders of magnitudes. The limit concentration required for the 2-AP aziridination of these lipids was also investigated and the C═C position diagnostic ions could be observed at 40-100 nM with good signal to noise ratio.

### Example 5

Identification and Quantification of Lipids with Multiple C═C Bonds

Polyunsaturated fatty acids (PU FAs) have at least two C═C bonds within their structures and are major constitutes of many lipid species across different categories. To further investigate the general applicability of the developed AAPI tags in the analysis of lipids that contain multiple C═C bonds within one FA chain, FAEE 18:2 (Δ9, Δ12) was firstly employed. Previous experiments with CE 18:1 lipid indicated that both of the two C═C bonds could be completely converted to the aziridines within 2 hrs when 15 mM 2-AP reagent was used. However, only ˜22% di-aziridine products were obtained in the derivatization of 100 μM FAEE (Δ9, Δ12) under the identical experimental conditions for 24 hrs, and ˜70% FAEE (Δ9, Δ12) was converted to mono-aziridines. It was reasoned that the incomplete conversion of FAEE (Δ9, Δ12) resulted from the steric hindrance of the two closely distributed C═C bonds within one FA chain. CID fragmentation of mono-aziridine products of FAEE (Δ9, Δ12) at m/z 401.4 generated fragments at m/z 217.2 & 277.2 and 177.1 & 317.3 from the cleavage of the aziridine ring at the Δ9 and Δ12 positions. The intensities of the two sets of diagnostic ions were comparable to each other, suggesting that the two C═C bonds have similar reactivities. Similarly, 85% di-aziridine products and ˜4% tri-aziridine and were obtained in the derivatization of 100 μM CE (Δ9, Δ12). CID-MS² of the di-aziridines of CE (Δ9, Δ12) at m/z 417.4 (z=2) generated two major fragments at m/z 461.3 and 373.3, corresponding to [Chol+2-AP+H—H₂O]⁺ and 2-AP mono-labeled fatty acyl [FA 18:2+2-AP+H]⁺ ions. Further CID of the fragment ions at m/z 373.3 yields diagnostic ions at m/z 217.2 & 249.1 and 177.1 & 289.2 to confirm the original C═C bonds are located at the Δ9 and Δ12 positions while the ion pairs of 177.1 & 289.2 had higher ion abundances. It was believed that the C═C bond at the Δ12 positions might suffer less steric hindrance from the cholesteryl ring. These results indicated that the mono-aziridines are dominant products for FA with two C═C bonds. Nevertheless, the identification of lipid C═C bond positions and good quantification accuracy could also be achieved using the dominant mono aziridines products.

In addition to FAEE (Δ9, Δ12) and CE (Δ9, Δ12), lipid standards with more than two C═C bonds were also investigated. In the analysis of FA 18:3 (Δ9, Δ12, Δ15), it was found that ˜68% of di-aziridine products were obtained with the existence of ˜30% of by-products, which corresponded to the elimination products of tri-aziridines. Interestingly, the fragmentation pathways of the di-aziridines of FA 18:3 (Δ9, Δ12, Δ15) differ from their mono-aziridines. CID of its major di-aziridine products at m/z 463.3 yields fragment ions at m/z 147.1, 187.1, 261.2 and 301.2, corresponding to the cleavage of nearly carbon-carbon single bond (C—C bond) connecting to the aziridine ring. These diagnostic ions indicated that the C═C bonds are located at the Δ15, Δ12, Δ9, and Δ12 positions, respectively. Among them, ions at m/z 147.1 and m/z 261.2 are of higher abundances. As for FA 18:3 (Δ6, Δ9, Δ12), the diagnostic ions were found at m/z 189.1 (Δ12), 219.2 (Δ6), 229.2 (Δ9) and 259.2 (Δ9) accordingly, and ions of m/z 189.2 and 219.2 are dominant.

To further investigate if the di-aziridines of FA 18:3 and their fragment ions can be used for quantification, a series of lipid mixtures of FA 18:3 (Δ9, Δ12, Δ15) and FA 18:3 (Δ6, Δ9, Δ12) was then prepared in the two protocols: i) the total Concentration of FA 18:3 was kept constant at 100 μM with the mol % of FA 18:3 (Δ9, Δ12, Δ15) varied from 0%, 20%, 40%, 60%, 80% to 100% and (ii) FA 18:3 lipid solution was prepared at the concentration of 0.4 μM, 2 μM, 10 μM, 50 μM, 100 μM to 250 μM, which included 50 mol % of FA 18:3 (Δ9, Δ12, Δ15) and FA 18:3 (Δ6, Δ9, Δ12). These solutions were derivatized using 2-AP-[d0] (15 mM), while a solution containing 25 μM FA 18:3 (Δ9, Δ12, Δ15) and 25 μM FA 18:3 (46, 49, 412) was labelled by 2-AP-[d4] (15 mM) as control. After 24 hr reaction, the 2-AP-[d0] and 2-AP-[d4] derivatized lipid aziridines were mixed in 1:1 ratio for nESI-MS/MS analysis with 0.1% formic acid. When the intensity ratios of major diagnostic ions for FA 18:3 (Δ9, Δ12, Δ15)-2-AP-[d0] (m/z 147.1 & 261.2) and FA 18:3 (Δ9, Δ12, Δ15)-2-AP-[d8] (di-aziridines, m/z 151.1 & 265.2) were plotted against their molar ratios, both of two linear relationships obtained had a slope very close to 1.00 and R-square values as 0.9982 and 1.000, respectively. Similar results for FA 18:3 (Δ6, Δ9, Δ12) (m/z 189.1 & 219.2 with 2-AP-[d0] and m/z 193.2 & 223.2 with 2-AP-[d4]) were also achieved either when the FA 18:3 was 100 μM with isomer mol % varied or when the FA 18:3 isomer mixtures were prepared across more than two orders of magnitude dynamic range. Moreover, during the quantitative analysis of FA 20:4 (Δ5, Δ8, Δ11, Δ14), and FAEE 20:4 (Δ5, Δ8, Δ11, Δ14), CE 20:4 (Δ5, Δ8, Δ11, Δ14), CID of the di-aziridines follow similar fragmentation patterns of FA 18:3 and good quantification accuracy can be achieved as well using their major diagnostic ions. These results indicated that the A API tags could successfully achieve the identification of lipid with multiple C═C bonds in a FA chain and quantification.

The precise structure characterization and quantification of lipid extract solutions have proved to be difficult since it often contains various lipid species as well as multiple structural isomers. Therefore, two artificial lipid mixture solutions in 1:2 concentration ratio were then prepared to evaluate the feasibility of the AAPI tags in the analysis of lipid mixtures, which contained FA 16:1, FA 18:1, FA 18:3 FAEE 18:1, PC 18:1-18:1, CE 18:1, and TAG 18:1-18:1-18:1. The two solutions were labelled using the 2-AP-[d0] and 2-AP-[d4] tags separately and then mixed in 1:1 ratio for nESI-MS/MS analysis. The measured intensity ratios of parent ions and the diagnostic ions upon CID for the eight lipids were around 2, indicating that the concentration ratios of lipids in the two solutions could be read out directly using the intensity ratios with high accuracy.

### Example 6

HPLC-MS Analysis of Yeast Lipid Extract Coupled with AAPI Tagging

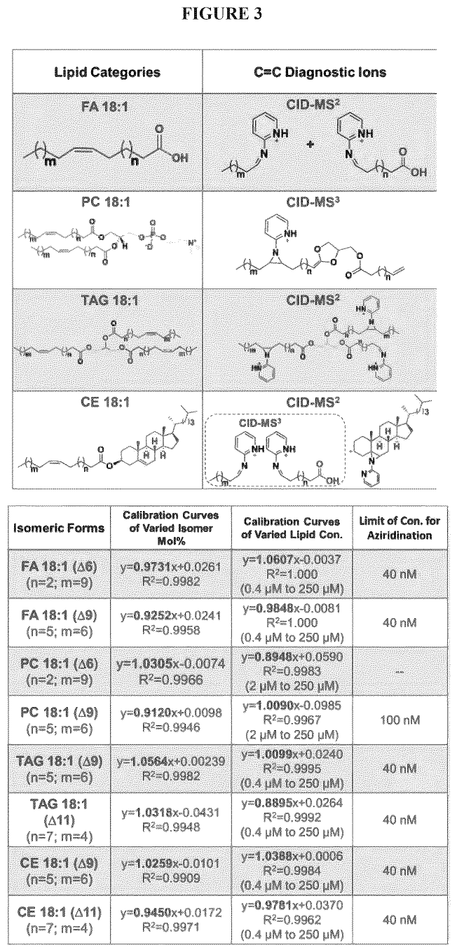

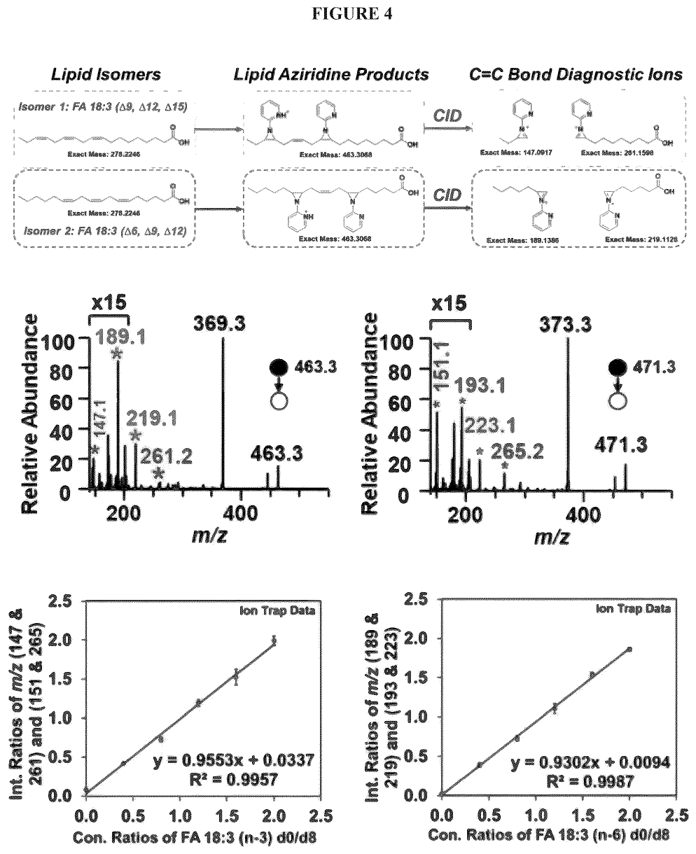

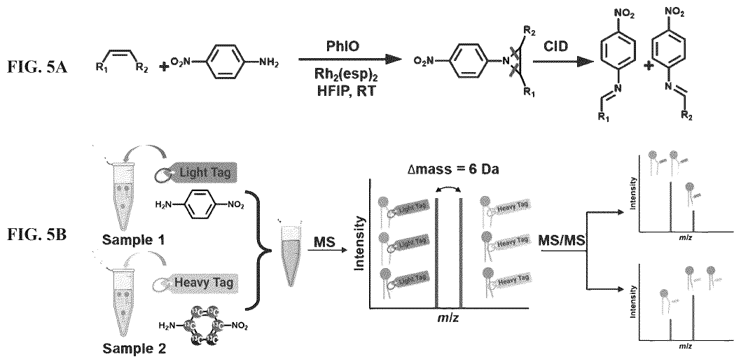

Liquid chromatography (LC)-MS have been extensively used in the analysis of complex lipid mixtures where the lipid species could be separated based on the properties of their polar headgroups or hydrophobic fatty acyl chains. Thus, LC-MS-based lipidomic workflow could significantly simplify the sample matrix and minimize the mass overlap owing to the existence of various isobaric and isomeric lipids. To evaluate the feasibility of merging the AAPI tags onto the LC-MS platforms for large-scale lipidomic study, a yeast lipid extract was first employed. In the experiment, two yeast lipid mixture solutions of 200 μM and 100 μM were labeled using 2-AP-[d0] (light tag) and 2-AP-[d6] (heavy tag), respectively and then mix together for RPLC-MS analysis. Taking lipid PC 18:0_18:1 as an example, the retention time of its 2-AP derivatized lipids was found at 18.90 min, while 2-AP itself was found before the reaction has the retention time at 20.72 min under the same RPLC-MS condition (FIGS. 3A-3B). CID of the 2-AP-[d0] and 2-AP-[d6] derivatized PC 18:0_18:1 produced the diagnostic ions at m/z 571.5 and m/z 575.5. The extracted ion chronograms (EICs) of the two diagnostic ions identified the Δ9 positions of the C═C bonds in PC 18:0_18:1. Meanwhile, their peak area ratio was 1.9706±0.2059, which accurately represented the concentration ratio of PC 18:0_18:1 between the two yeast lipid mixture solutions. Similarly, the other 24 lipid species including FA, PC, PI, PE, PA and PG were identified with their C═C bond positions resolved and their concentration ratios between the two samples were truly reflected by directing reading out their diagnostic ion peak areas. These results proved the potential of coupling the AAPI tags with the RPLC-MS for large-scare lipid analysis.

### Example 7

### Materials and Methods of Examples 8-11

### Lipid Nomenclature

Notations for lipids were adopted from LIPID MAPS. The position of C═C in an aliphatic chain is either defined by the Δx nomenclature or n-x nomenclature, where Δx nomenclature represents the carbon counts from the alpha carbon of the fatty acyl chain and n-x nomenclature counts from the methyl terminus. For example, PC 16:0/18:1(9Z) means phosphatidylcholine with a 16-carbon fatty acyl chain on sn-1 position and an 18-carbon fatty acyl chain on sn-2 position that carry a carbon-carbon double bond on Δ9 position and the bond geometry is Z. The sn position and the Z/E stereo-configurations of the carbon-carbon double bond cannot be assigned based on the method, and they were not shown for lipids that identify from biological samples.

### Cell Lines, Cell Culture and Chemicals

Human lung cancer cell line A 549 was obtained from the American Type Culture Collection and human breast cancer cell line MDA-MB-231 was a gift. A 549 cells were cultured in Dulbecco's modified Eagle's medium with L-Glutamine and 4.5 g/L Glucose (DM EM, Corning 10017CV) supplemented with 10% heat-inactivated fetal bovine serum (FBS, Gibco 10438026) and 1% penicillin-streptomycin (PS, 5000 IU Penicillin, 5000 μg/mL Streptomycin, Corning MT30001CI). MDA-MB-231 cells were cultured in Ham's F-12K (Kaighn's) Medium (F-12K, Gibco 21127022) with 10% FBS and 1% PS. Both cell lines were cultured in 5% CO₂ in a humid atmosphere at 37° C. For the inhibition experiment, low serum conditions (0.5% FBS for MDA-MB-231 and 2% FBS for A 549) were applied. Ethyl oleate (FAEE) and fatty acids (FA) were purchased from Sigma-Aldrich (MO, USA), phosphatidylcholines (PC), bovine heart extract, and yeast extract were purchased from Avanti Polar Lipids (AL, USA). Cholesteryl ester (CE) and triacylglycerol (TAG) were purchased from Nu-Chek Prep (MN, USA). ¹³C 6-4-nitroaniline and 5(Z)-Dodecenoic Acid were purchased from Caymen Chemical (MI, USA). Iodosobenzene (PhIO) was purchased from Aaron Chemicals (Jiangsu, China). Bis [rhodium (α,α,α′,α′-tetramethyl-1,3-benzenedipropanoic acid)] (Rh₂(esp)2) was purchased from Ambeed, Inc. (IL, USA). CAY 10566 and SC-26196 were purchased from MedChemExpress LLC (NJ, USA). Hexafluoro-2-propanol (HFIP) was purchased from CHEM-IM PEX (IL, USA). Phosphate-buffered saline (PBS) was purchased from Gibco (MA, USA). 2,2,4-Trimethylpentane, ammonium formate, and formic acid was purchased from Fisher Scientific (NH, USA). Dimethyl sulfoxide (DMSO) was purchased from Corning (NY, USA). Other solvents including water (H₂O), acetonitrile (ACN), isopropanol (IPA), dichloromethane (DCM), methanol (MeOH), methyl tert-butyl ether (MTBE), and chloroform are HPLC grade and were purchased from Sigma-Aldrich (MO, USA).

### Inhibition Experiment and Lipid Extraction

Cancer cells were seeded in 100 mm petri dish (Thermo Scientific Nunc, 150350) at 2.2×10⁶ cells in 10% FBS medium and grown in a humidified environment at 37° C. with 5% CO₂. After 24 hr., the medium was aspirated, cells were washed with PBS and low FBS medium (0.5% FBS for MDA-MB-231 and 2% FBS for A 549), supplemented with 1 μM CAY 10566 or 1 μM and 24 μM SC-26196 dissolved in DMSO. The same volume of DMSO was added for the control. Treatment was carried out for 72 hr. during which cells were grown in a humidified environment at 37° C. with 5% CO₂. Cells were trypsinized (MilliporeSigma SM 2003C), washed with cold PBS, and counted with an automated cell counter (Countess 3, Invitrogen, Thermo Fisher Scientific). After counting, cells were collected in microcentrifuge tubes by centrifuge. Internal standard 5(Z)-Dodecenoic Acid was added to the microcentrifuge tubes and lipids were extracted following an MMC protocol. In brief, 1000 μL of a MeOH:MTBE:chloroform (MMC) 1.33:1:1 (v/v/v) mixture were added in microcentrifuge tubes with cell pellets, vortexed for 30 s, and shaken for 20 min at 1000 rpm at room temperature. Next, the plates were centrifuged (5 min at 3000 rpm, room temperature) and the solution was collected and dried under nitrogen flow. The saponification and FA extraction followed a protocol well known in the art. Briefly, the extracted lipids were saponified in 500 μL ACN: 15% KOH (50/50, v/v) at 60° C. for 60 min. The solution was acidified with 1 M HCl (1 mL). The hydrolyzed total FA s were extracted twice with 1.5 mL 2,2,4-Trimethylpentane each time. The organic layer was collected, dried using rotary evaporators (IKA) for further N-aryl aziridination.

### General Procedures for N-Aryl Aziridination

Lipid standards, bovine heart and yeast extract, and total FA extract from cancer cells were dissolved in DCM:HFIP (20/80, v/v) and mixed with either 4-nitroaniline or ¹³C 6-4-nitroaniline (3 mM), PhIO (3 mM), and Rh₂(esp)₂ (0.5 mM). The reaction mixture was stirred at room temperature for 20 hours. Then the two reaction mixtures (4-nitroaniline and ¹³C6-4-nitroaniline) were mixed in a 1 to 1 volume ratio, diluted with ACN (to a final concentration of 25 μM) and 0.1% formic acid was added for nano-ESI direct infusion or diluted with ACN:IPA (1/1, v/v) for HPLC-MS analysis.

### Instrumentation and Details of Data Acquisition

Direct infusion was carried out on a Thermo Scientific LTQ XL (Thermo Fisher Scientific) using nano-ESI. The nano-ESI tips were pulled from borosilicate glass capillaries (1.5 mm o.d. and 0.86 mm i.d., purchased from World Precision Instruments, Sarasota, FL, USA) using P-1000 micropipette puller (Sutter Instrument, Novato, CA). A platinum wire was inserted into the tip to provide efficient electrical contact with the solution to initiate the electrospray. The following MS parameters were used for nano-ESI data acquisition: in full scan MS, a mass range of m/z 200-1500 was used with a scan rate of 2 micro scans and max inject time is 200 ms. The spray voltage was between 1.5 KV to 3.0 kV. Capillary voltage was set at 44 V and tube lens was set at 85 V in positive ion mode. The capillary temperature was 275° C. CID normalized collision energy between 18 to 28 and an isolation window between 1.5 to 3.0 were used for MS². Source fragmentation energy of 35 V was applied for some experiments to remove the unwanted adductions.

HPLC-MSⁿ analysis of lipid standard TAG 18:1(9Z)/18:1(9Z)/18:1(9Z), PC 18:1(9Z)/18:1(9Z), bovine heart extract, and yeast extract was carried out on a vanquish UHPLC system (Thermo Fisher Scientific) coupled with an Orbitrap Velos Pro mass spectrometer (Thermo Fisher Scientific). An aliquot of 5 μL of the sample was injected onto the Accucore C30 column (Thermo Fisher Scientific, C30, 2.1 mm×150 mm, 2.6 μm). The mobile phase used was ACN/H₂O (60/40, v/v) (solvent A) and IPA/ACN (90/10, v/v) (solvent B), both containing 10 mM of ammonium formate and 0.1% formic acid. The column separation was carried out at 40° C. with a flow rate of 0.2 mL/min. The elute gradient was as follows: 30% B at 0-3 min (to waste), 30-43% B at 3-8 min, 43-50% B at 8-9 min, 50-90% B at 9-18 min, 90-99% B at 18-30 min, 30% B at 30-35 min. H-ESI source was used for the analysis and the parameters are: sheath gas 35 arb, auxiliary gas 10 arb, sweep gas 1 arb, spray voltage 3.5 kV, capillary temperature 350° C. The parameters of MS for the full MS are as follows: S-lens RF Level 60%, analyzer FTMS, resolution 60,000, data type profile. For MSⁿ analysis, the parameters are as follows: detector Ion Trap, isolation window (m/z) 3.0, CID collision energy 35%, CID activation time 10 ms, activation Q 0.25, RF lens 60%.

Orbitrap Fusion Tribrid mass spectrometer (Thermo Fisher Scientific) coupled with vanquish UHPLC system (Thermo Fisher Scientific) was used for total FA analysis of cancer cells. An aliquot of 5 μL of the sample was injected onto the Accucore C18 column (Thermo Fisher Scientific, C18, 2.1 mm×150 mm, 2.6 μm). The mobile phase used was ACN/H₂O (60/40, v/v) (solvent A) and IPA/ACN (40/60, v/v) (solvent B), both containing 10 mM of ammonium formate and 0.1% formic acid. The column separation was carried out at 60° C. with a flow rate of 0.5 mL/min. The elute gradient was as follows: 30% B at 0-0.4 min (to waste), 30-45% B at 0.4-0.9 min, 45-52% B at 0.9-1.1 min, 52-58% B at 1.1-1.8 min, 58-66% B at 1.8-2.5 min, 66-70% B at 2.5-3.1 min, 70-75% B at 3.1-4 min, 75-97% B at 4-4.5 min, 97% B at 4.5-6 min, 30% B at 6.1-7 min. H-ESI source was used for the analysis and the parameters are: spray voltage 3.5 kV, sheath gas 60 arb, auxiliary gas 15 arb, sweep gas 2 arb, ion transfer tube temperature 350° C., vaporizer temperature 400° C. The parameters of MS for the full MS are as follows: detector Orbitrap, resolution 60,000, use quadrupole isolation, scan range 250-1000, RF lens 60%, normalized A GC target 50%, maximum injection time 100 ms, microscan 1, data type profile, positive. MS parameters for the MS² acquisition are as follows: isolation mode quadrupole, detector Ion Trap, isolation window (m/z) 1.5, CID collision energy 35%, CID activation time 10 ms, activation Q 0.25, RF lens 60%, A GC target standard, maximum injection time auto, microscan 1, datatype centroid.

For the HPLC-MSⁿ analysis of bovine heart extract, yeast extract, and total FA from cancer cells, data-dependent acquisition (DDA) was first used to obtain the target list of aziridine-lipids in the sample. The MS setting for DDA are as follows: number of dependent scans 10, dynamic exclusion on, exclude after 1 time, exclusion duration 3 s, mass tolerance low/high 5 ppm, exclude isotope true, isolation mode quadrupole, isolation window (m/z) 1.5, CID collision energy 35%, CID activation time 10 ms, activation Q 0.25, detector Ion Trap, AGC target standard, maximum injection time 35 ms, microscan 1, datatype centroid.

The analysis of lipid aziridines was performed using a list of expected CE aziridines based on the list of the native CE lipids obtained above. A cycle of five scan events was used including one full MS scan and four CID tandem MS scans with two MS² using the normalized collision energy of 35-40 and two MS³ scans using the normalized collision energy of 35. The isobaric labeled CE lipids were analyzed using HCD tandem scans with 60 normalized collision energy under the non-data-dependent acquisition mode. Full mass spectra were collected at a resolving power of 30,000, while tandem mass spectra were obtained at a resolving power of 15,000. Targeted HPLC-MS2 analysis was used after obtaining the aziridine-lipid list in a start/end time mode.

### Data Analysis

The data acquired from nano-ESI was analyzed on the X calibur Qual Browser. The S/N for the limit of concentration for aziridination was obtained using X calibur FreeStyle. HPLC-MSⁿ data acquired using DDA was analyzed using a home-written Python script. The script first searches for ion pairs differing by characteristic ¹³C mass increments (e.g., 6, 12, 18 Da) in full MS with intensity ratios ranging from 0.1% to 100%. Then it uses tandem MS to confirm the class of lipids. The m/z tolerance of searching ion pairs in full MS is 0.005 and the intensity threshold is 10,000. Neutral loss 138 Da was used for general aziridine-lipid identification. Determination of lipid class is using characteristic fragment ions or neutral loss: 184 Da for PC, 505 Da for CE, −172 Da for PG, −141 Da for PE, −260 Da for PI, −97 Da for PA. The m/z tolerance for tandem MS is 0.2 and the intensity threshold is 1000. For the targeted HPLC-MSⁿ analysis for relative quantification, a home-written Python script (version 3.11.5) was used to extract the diagnostic ions intensity in the tandem MS within certain user defined retention time range. The m/z tolerance for diagnostic ions is 0.2.

For determination of relative FA abundance change, the diagnostic ion ratios in tandem MS and the sum level FA ratios in full MS were normalized to an internal standard (5(Z)-Dodecenoic Acid) and the cell number for cell extracts.

### Example 8

### N-Aryl Aziridination of Unsaturated Lipids and its Use in Mapping C═C Bond Positions

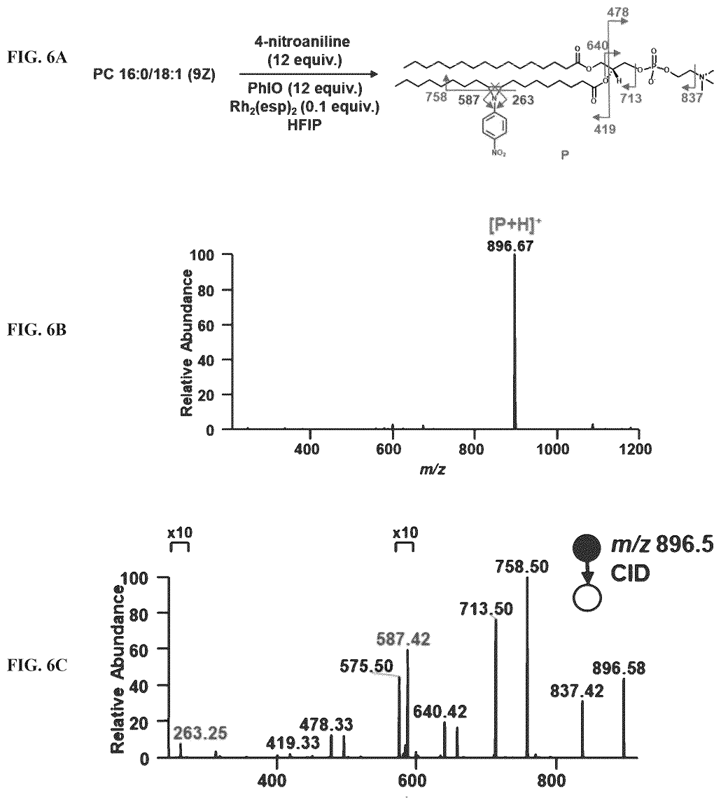

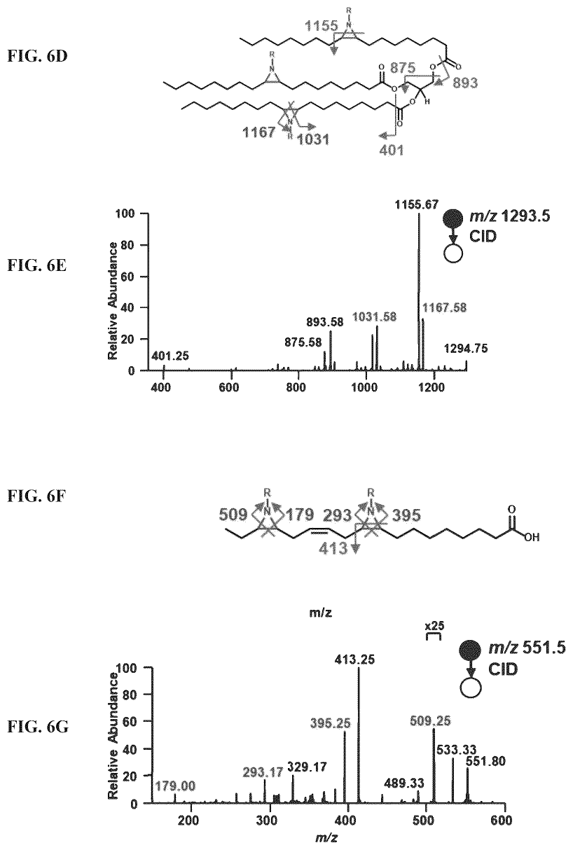

Nano-ESI and a series of lipid standards were employed (Table 1) to evaluate the labeling efficiency of N-aryl aziridination. First, PC 16:0/18:1(9Z) was examined, as its native form is readily observed in MS, facilitating the calculation of labeling yield (FIG. 6A). Under room temperature conditions with 12 equivalents of PhIO and 4-nitroaniline and a rhodium catalyst, complete conversion to the aziridine-lipid (m/z 896.67) was achieved within 2 hours (FIG. 6B). No native PC (m/z 760.59) remained, indicating a 100% labeling yield.

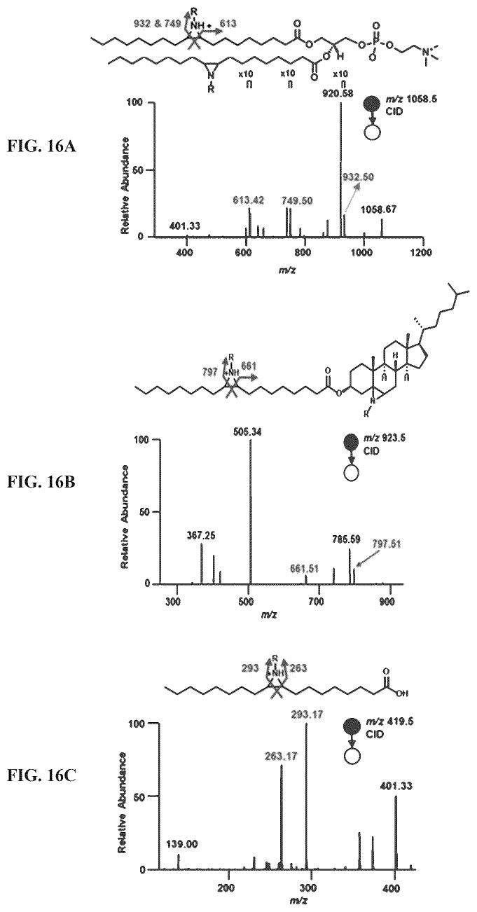

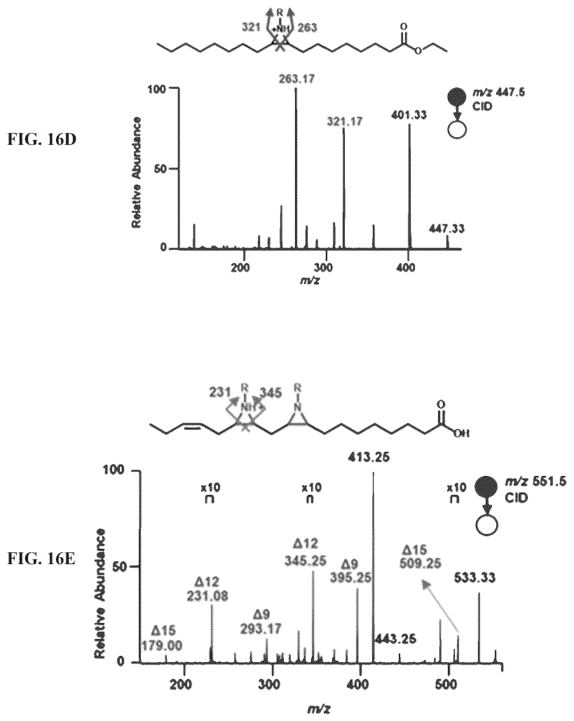

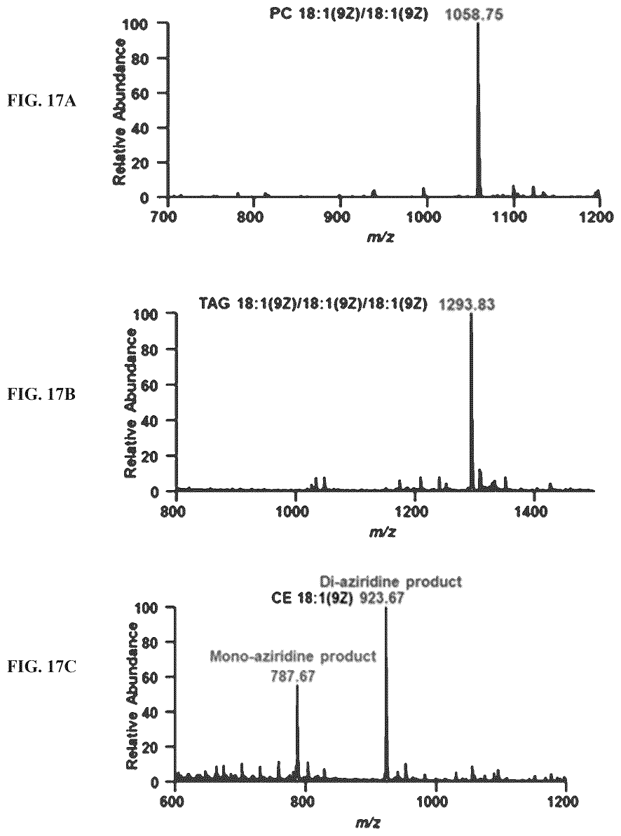

Encouraged by this result, the study was extended to lipids with multiple C═C bonds. For PC 18:1(9Z)/18:1(9Z), the same reaction conditions yielded only the di-aziridine product (m/z 1058.75), with no unreacted lipid detected (m/z 786.60) (FIG. 16A). Reducing the equivalents of PhIO and 4-nitroaniline below 12, however, produced both the mono-(m/z 922.75) and di-aziridine forms. Next, triacylglycerides (TAGs) and cholesterol esters (CEs) were investigated, which typically have low ionization efficiencies in MS. The TAG 18:1(9Z)/18:1(9Z)/18:1(9Z), containing three double bonds on each fatty acyl chain, underwent complete tri-aziridine formation (m/z 1293.67) after 20 hours under the same conditions (FIG. 16B). By contrast, CE 18:1(9Z), with one C═C on the fatty acyl chain and another on the sterol ring, primarily formed the di-aziridine product (m/z 923.58) after 20 hours. A small amount of the mono-aziridine product (m/z 787.58) persisted even after 24 hours, likely because of steric hindrance at the sterol ring (FIG. 16C). Lipids with multiple C═C bonds on a single fatty acyl chain were also tested, using FA 18:3 (9Z, 12Z, 15Z) as a model. Approximately 45% of the di-aziridine product (m/z 551.50) was obtained, while around 55% of the labeled fragment (m/z 413.42) resulted from tag detachment (FIG. 16D). Even after 24 hours, no tri-aziridine product was observed, presumably due to steric constraints from closely spaced double bonds.

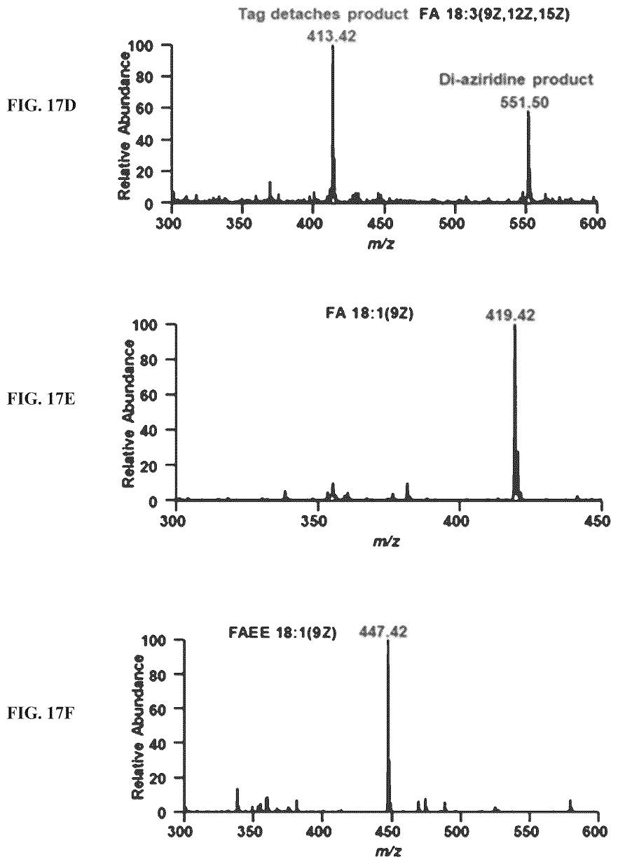

Tandem MS confirmed the capability of the aziridine tag to elucidate lipid structures, including precise C═C locations. As expected, the aziridine ring opens upon CID to generate diagnostic ions (DIs) for localizing double bonds, an outcome not possible with native lipids. For PC 16:0/18:1(9Z), aziridine fragmentation yielded a pair of DIs at m/z 263 and 587, pinpointing the Δ9 double bond (FIG. 6A, FIG. 6C). Additional fragments included m/z 837 and 713 (partial and complete choline headgroup loss), m/z 419 (unsaturated fatty acyl chain 18:1), m/z 478 and 640 (loss of fatty acyl chains), and m/z 758 (loss of the tag). Collectively, these fragments reveal the lipid class, molecular structure, and C═C position. Similarly, for TAG 18:1(9Z)/18:1(9Z)/18:1(9Z), aziridine fragmentation produced DIs at m/z 1031 and 1167 for the Δ9 positions, as well as peaks at m/z 893 and 875 (fatty acyl chain loss), m/z 401 (fatty acyl chain with subsequent water loss), and m/z 1155 (loss of the tag) (FIG. 6D, FIG. 6E). C═C DIs were also detected for PC 18:1(9Z)/18:1(9Z), CE 18:1(9Z), FA 18:1(9Z), and FAEE 18:1(9Z) (FIG. 17A-FIG. 17D). In the case of FA 18:3 (9Z, 12Z, 15Z), tandem MS of the di-aziridine product indicated that derivatization predominantly occurred at the Δ9 and Δ15 bonds, with corresponding DIs at higher abundance (FIG. 6F, FIG. 6G), although low-intensity DIs for the Δ12 position (m/z 231 and 345) were also observed (FIG. 17E).

### Example 9

### ¹³C Isotopic Labeling for Relative Quantification Using N-Aryl Aziridination

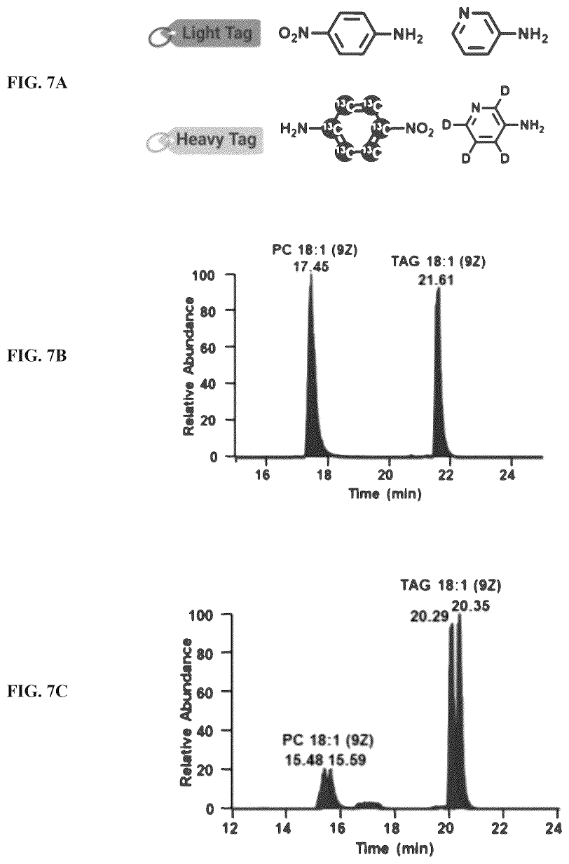

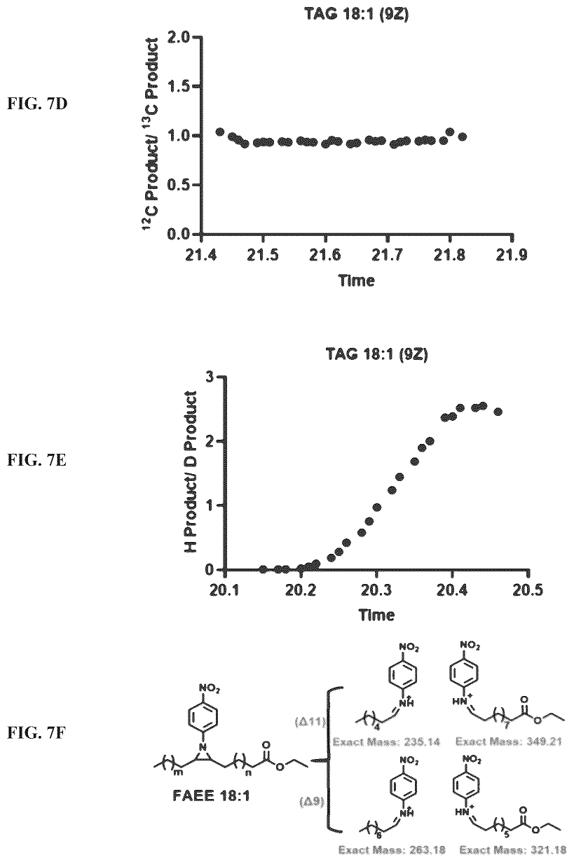

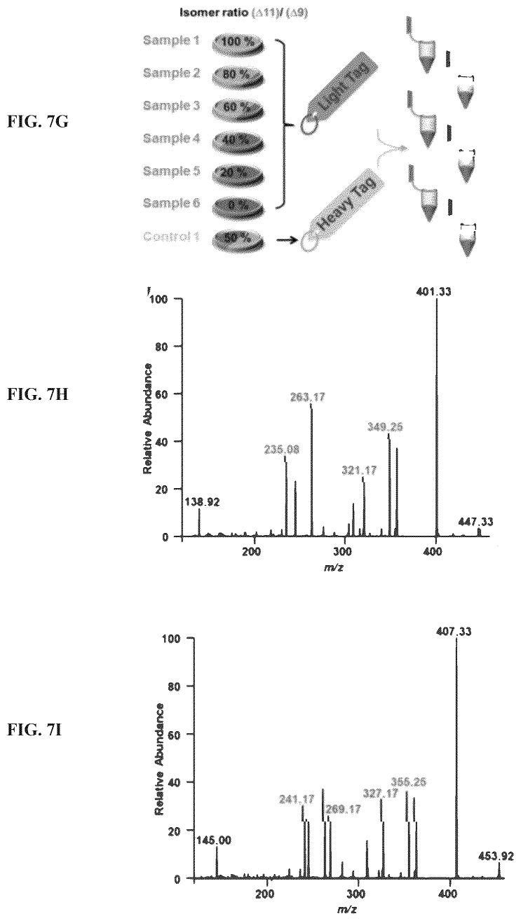

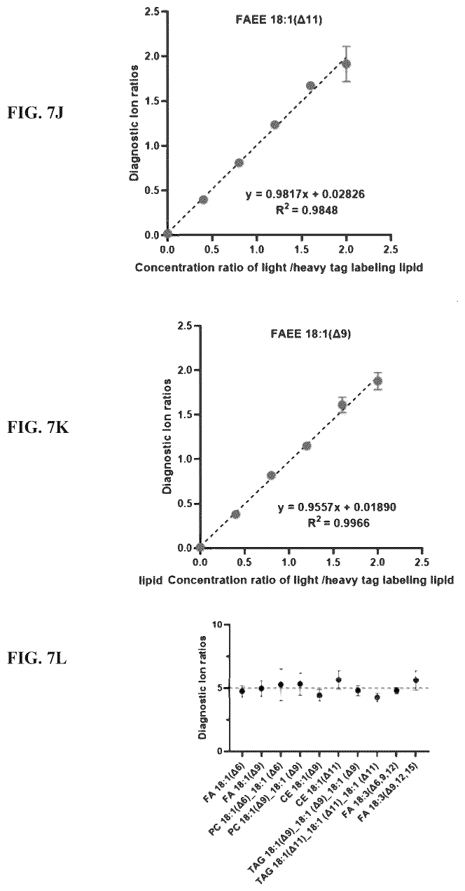

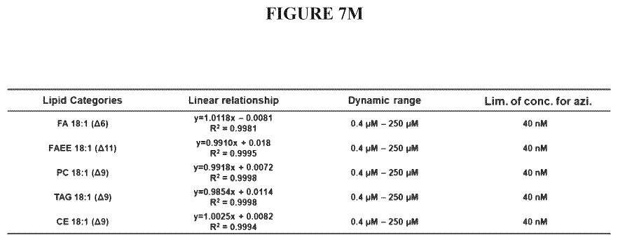

LC-MS has been extensively applied to the analysis of complex lipid mixtures, wherein lipids are separated based on the properties of their polar head groups or hydrophobic fatty acyl chains. In LC-based stable isotope labeling methods, deuterium is less favored because varying the number of deuterium atoms can shift retention times, due to differential interactions between hydrogen/deuterium and the reversed-phase stationary phase. This “deuterium isotope effect” arises from deuterium's larger mass and lower vibrational amplitude compared to hydrogen. To illustrate this effect, both TAG 18:1(9Z)/18:1(9Z)/18:1(9Z) and PC 18:1(9Z)/18:1(9Z) were labeled with “light” and “heavy” 2-aminopyridine at a 1:1 concentration ratio (FIG. 7A). The resulting chromatograms showed that the deuterium-labeled lipids eluted earlier, leading to ratio distortion in relative quantification (FIG. 7C, FIG. 7E) and necessitating full-peak integration to correct for retention-time shifts. The same experiment was performed using ¹³C-labeled 4-nitroaniline, observing nearly identical retention times for the light and heavy forms, with intensity ratios remaining close to 1.0 (0.95±0.03) throughout the peak (FIG. 7B, FIG. 7D). These findings suggest that ¹³C serves as a more suitable heavy atom for large-scale quantification, reducing both complexity and the number of spectra required for measurements.

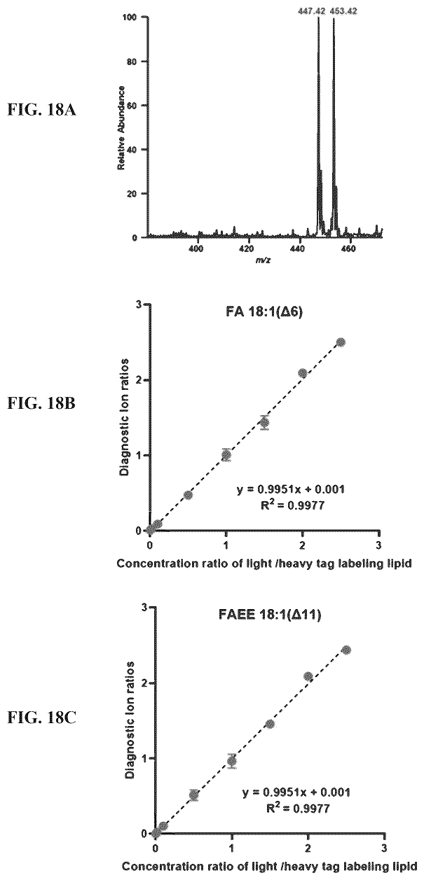

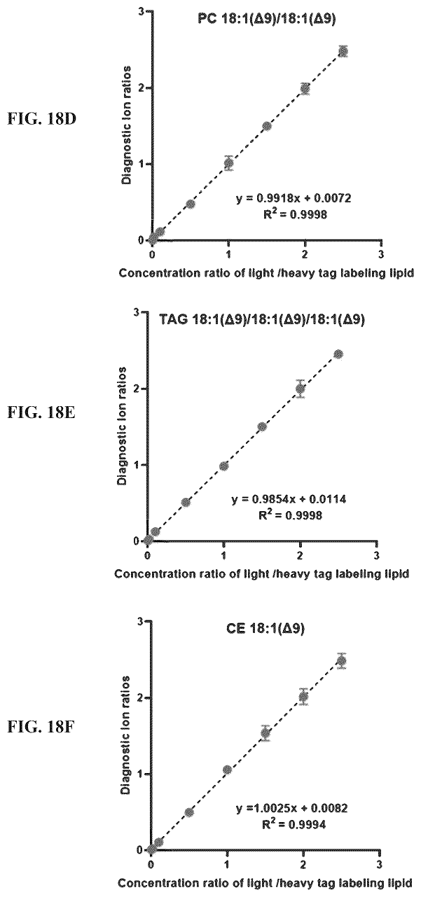

Next, fatty acid ethyl ester (FAEE) lipid standards were used to validate the quantification capability of the ¹³C-labeled mass tag for C═C positional isomers. Six samples of FAEE 18:1(9Z) and 18:1(11Z) were prepared at a total concentration of 100 μM and labeled with the light tag, varying the 9Z molar percentage from 0% to 100%. Meanwhile, a mixture containing 50 μM each of 9Z and 11Z was labeled with the heavy tag and served as the control (FIG. 7G). After aziridination, the six labeled samples were mixed 1:1 with the control prior to MS analysis. In the MS¹ spectra, two precursor ions were detected at m/z 447.42 (light) and m/z 453.42 (heavy) (FIG. 18A). CID of m/z 447.42 produced diagnostic ions at m/z 235.1 and 349.3 (indicating Δ11) and at m/z 263.2 and 321.2 (indicating Δ9) (FIG. 7H). Likewise, fragmenting m/z 453.42 yielded corresponding heavy-labeled DIs at m/z 241.2 and 355.3 (Δ11) and m/z 269.2 and 327.3 (Δ9) (FIG. 7I). Plotting the intensities of m/z 235.1 and 349.3 against those of m/z 241.2 and 355.3 revealed a strong linear correlation (R²=0.9848), a slope of 0.9817, and a y-intercept of 0.0283 (FIG. 7J). For the 49 isomer, the same approach gave R²=0.9966, with a slope of 0.9557 and a y-intercept of 0.0189 (FIG. 7K). Additional lipid classes were also examined in a 5:1 light-to-heavy labeling ratio, confirming relative quantification across diverse lipid categories (FIG. 7L).

To further establish the method's dynamic range, FAEE 18:1(11Z) concentration of 0.4, 2, 10, 50, 100, and 250 μM were derivatized with the light tag, while a 100 μM solution was derivatized with the heavy tag. After mixing them 1:1 (v/v) for MS analysis in 0.1% formic acid, the measured intensity ratios of diagnostic ions tracked closely with the expected ratios (R²=0.9995 and a slope of 0.99) (FIG. 18B). Similar tests with other lipid classes validated the method's applicability over two orders of magnitude (FIG. 7M, FIG. 18C-FIG. 18F). These results indicate that the N-aryl aziridination-based ¹³C isotopic labeling mass tag can provide relative quantification of lipids in different categories from two samples in a single experimental run with three orders of magnitude dynamic range. The limit concentration required for the N-aryl aziridination was also investigated and determined as 40 nM at which the signal remains 10-fold above the noise level with observable DIs in decent quality. Taken together, these results indicate that the N-aryl aziridination-based ¹³C isotopic labeling mass tag provides relative quantification of a broad range of lipids in a single run, featuring minimal signal distortion and a wide dynamic range.

### Example 10

HPLC-MS Analysis of Bovine Heart and Yeast Lipid Extract with Aziridination-Based ¹³C Mass Tags

Demonstrated herein is that N-aryl aziridination-based ¹³C isotopic labeling mass tag enables both the localization of C═C double bonds and relative quantification of unsaturated lipids. To further evaluate the method's applicability for large-scale lipid identification and quantification in complex biological samples, it was coupled with HPLC for quantitative structural lipidome analysis of bovine heart and yeast extracts. Light tag and heavy tag were used to separately label the lipid extract solutions of 200 μM and 100 μM (concentration ratio of 2) and mixed for reverse phase (RP) LC-MS analysis using data dependent analysis (DDA). A custom Python script (“paired aziridine-lipid search”) then generated a candidate lipid list for targeted HPLC-MSⁿ analysis for C═C positional isomer identification and relative quantification. In this semi-automated workflow, ion pairs differing by characteristic ¹³C mass increments (e.g., 6, 12, 18 Da) were detected in full MS with intensity ratios ranging from 0.1% to 100%. A tag-loss peak (−138 Da) in MS² confirmed each aziridine-lipid. Next, lipid class-specific fragment ions in MS² classified glycerophospholipids and CEs, while lipids lacking such ions (e.g., FAs, TAGs) were assigned as “general lipids” for manual verification. Because monounsaturated FAs and TAGs do not display the tag-loss peak in MS², these cases were verified individually.

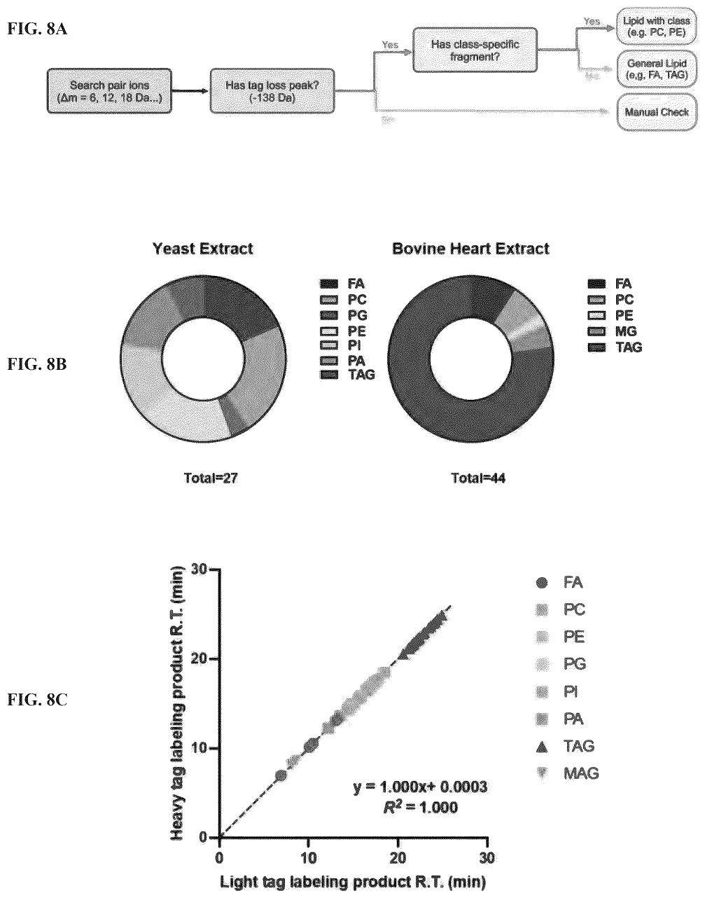

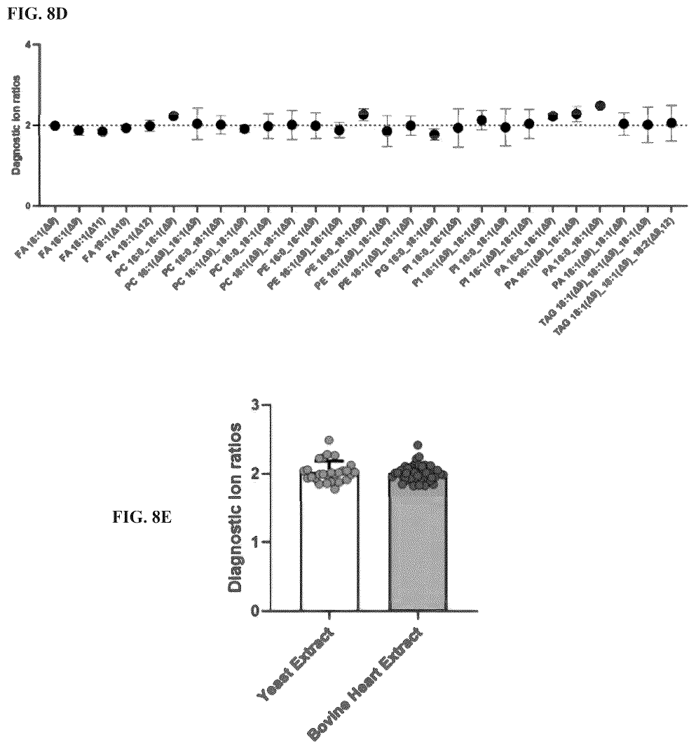

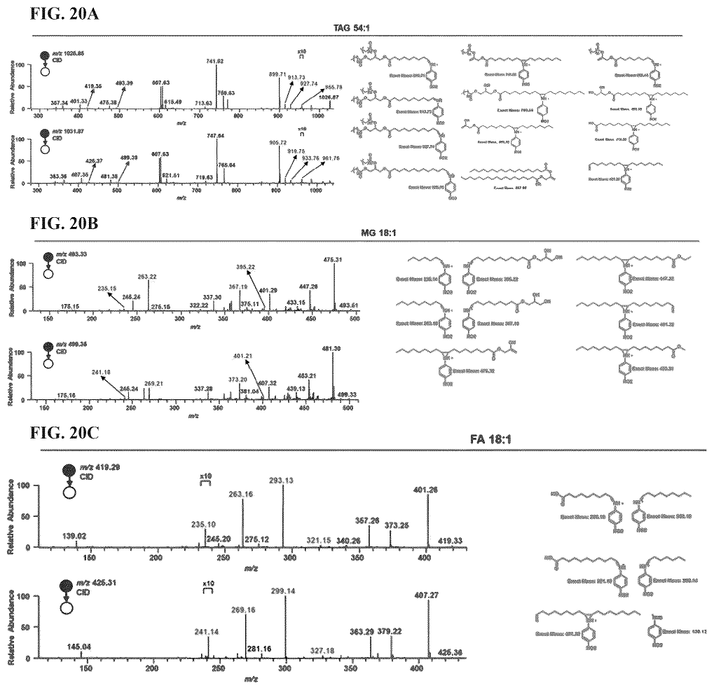

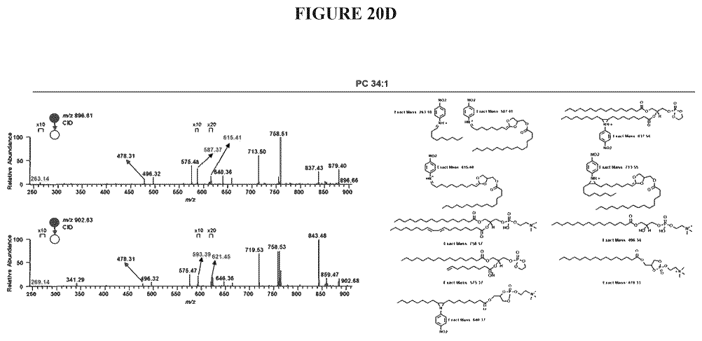

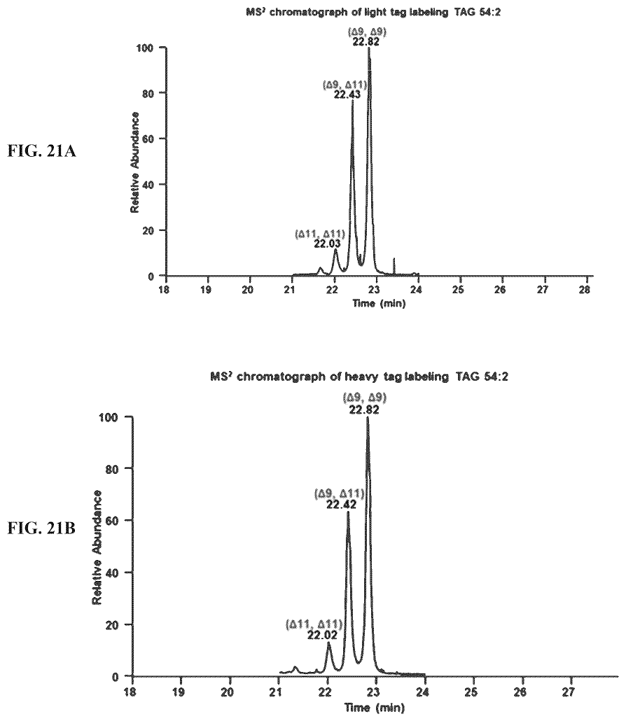

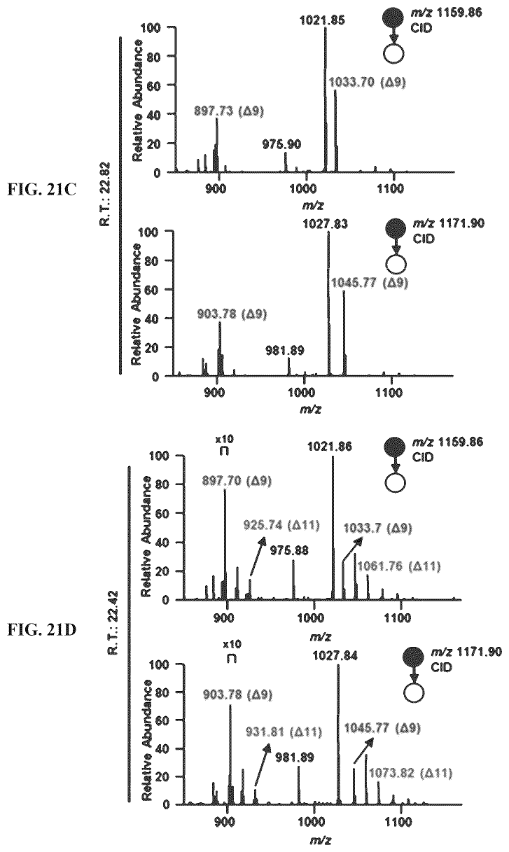

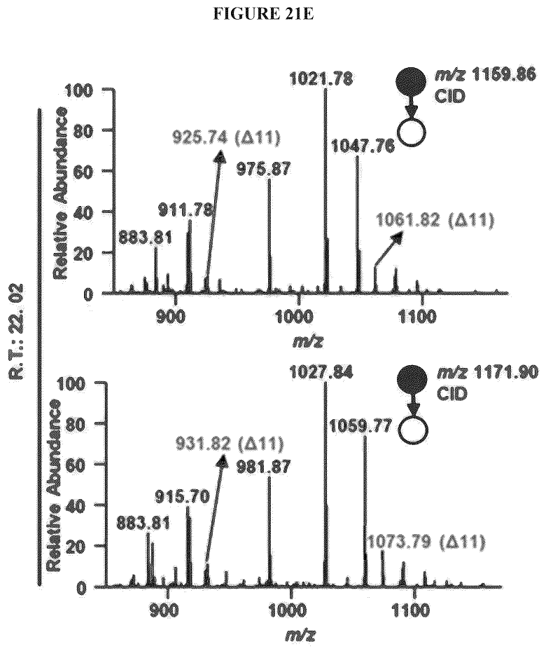

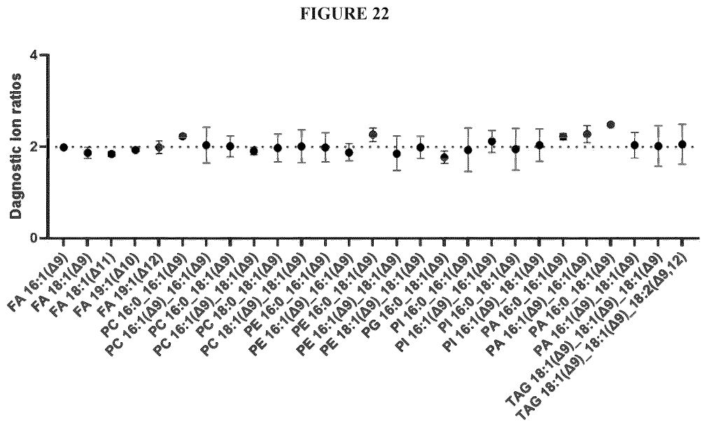

Through this workflow, 27 lipid C═C positional isomers in yeast extract and 44 in bovine heart extract were identified and relatively quantified (FIG. 8B, FIG. 20A-20D, FIG. 21A-21E). Plotting the retention times of light-tagged lipids against heavy-tagged lipids (FIG. 8C) showed a linear correlation (R²=1.000) with a slope of 1.000, confirming co-elution and demonstrating that ¹³C labeling does not shift chromatographic behavior in these complex samples. The DI ratios also matched the expected 2:1 concentration ratio, yielding average values of 2.03 and 2.01 with standard deviations of 0.27 and 0.17 in yeast and bovine heart extracts, respectively (FIG. 8D, FIG. 8E, FIG. 22).

### Example 11

### Relative Quantification of FAs in Human Cancer Cells Using Aziridination-Based ¹³C Mass Tags

Recent studies underscore the potential of targeting FA synthesis and modification pathways as a therapeutic strategy to inhibit tumor growth in various animal cancer models. Despite promising preclinical findings, clinical applications have faced challenges, partly because alternative pathways compensate for the pathway that is blocked by a given drug. Gas chromatography coupled with mass spectrometry (GC-MS) is commonly used to quantify FAs thus aid uncovering compensatory FA metabolism, yet it requires converting FAs into fatty acid methyl esters (FAMEs) and relies on internal standards for identification and quantification. Here, it is demonstrated that by applying the aziridination-based ¹³C isotopic labeling mass tag and FA desaturase inhibitor, the changes in FA abundance in human cancer cells can be identified and quantified without using internal standards-thereby shedding light on subsequent gene and protein expression studies.

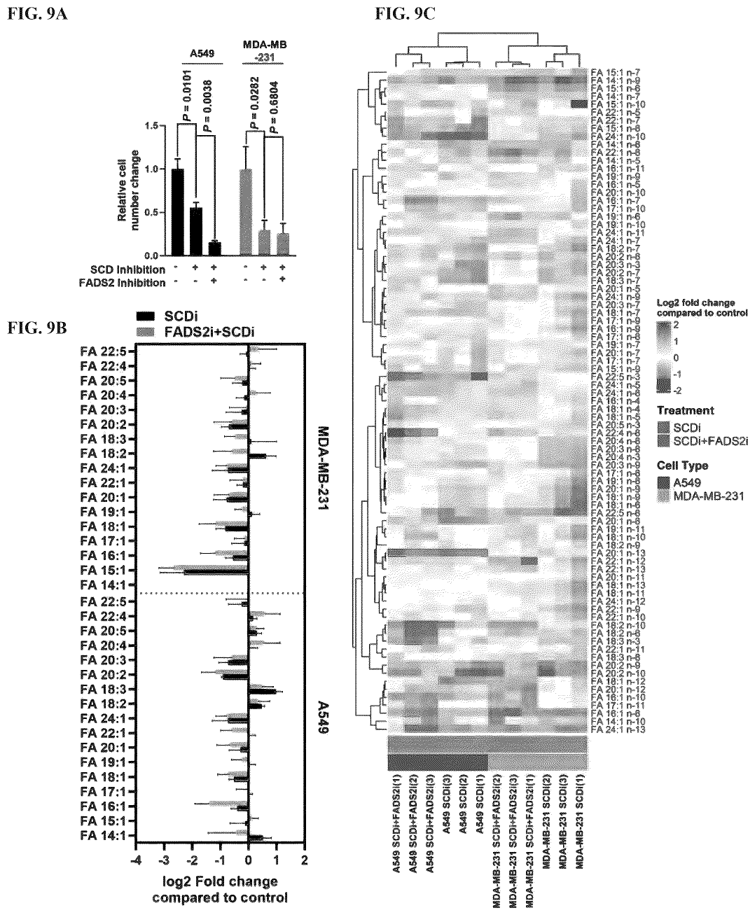

Stearoyl-CoA desaturase 1 (SCD1) and fatty acid desaturase 2 (FADS2) were selected as targets, with A 549 and MDA-MB-231 cancer cells as models. SCD1 is highly expressed in many cancers and has long been an attractive target in cancer therapy. Recently, it has been shown that some cancer cells such as A 549 can bypass SCD1 inhibition through a FADS2 compensatory pathway that maintains monounsaturated FA (MUFA) production, whereas MDA-MB-231 cells appear more reliant on SCD1 alone. First, both cell lines were treated with the SCD1 inhibitor (CAY 10566) under low extracellular FA availability. A 549 cells retained more than 50% of their proliferation compared to control, whereas MDA-MB-231 proliferation decreased to below 30% (FIG. 9A), consistent with previous studies. To test whether FADS2 compensation drives the differential response, both CAY 10566 and a FADS2 inhibitor (SC-26196) were employed, and it was observed that a significant reduction in A 549 proliferation (P=0.0038) occurred, whereas MDA-MB-231 was only modestly affected (FIG. 9A). Then, a ¹³C-labeling “heavy” tag for the DMSO control and a “light” tag for the SCD-inhibition or dual-inhibition groups was used, enabling a single LC-MSⁿ run to capture both sum-level and isomer-level FA changes. By comparing the intensities of paired ions in the full MS, it was observed that generally FA unsaturation decreased in both A 549 and MDA-MB-231 after enzyme inhibition (FIG. 9B).

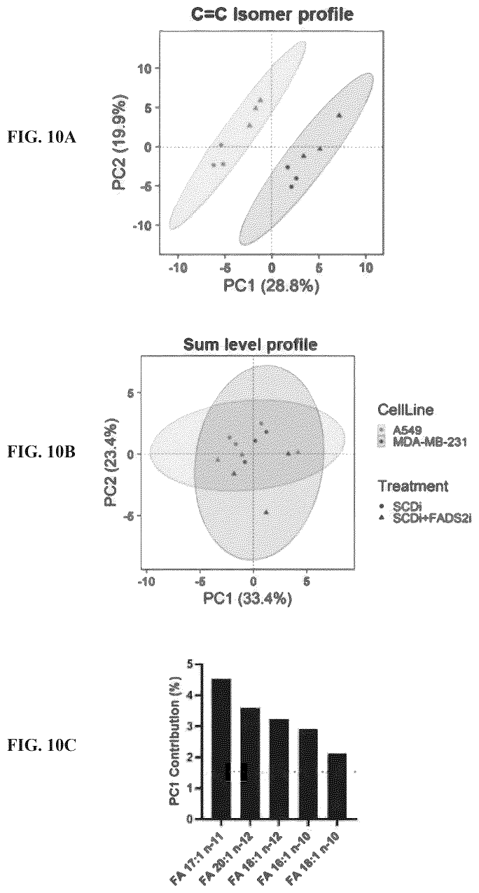

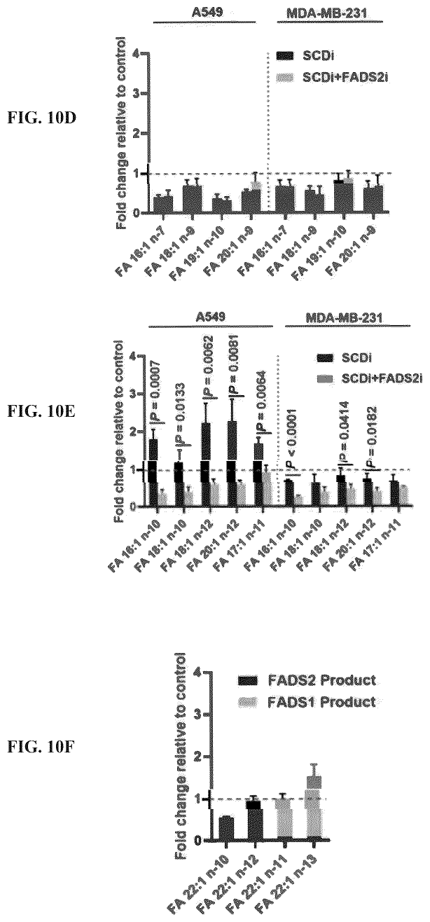

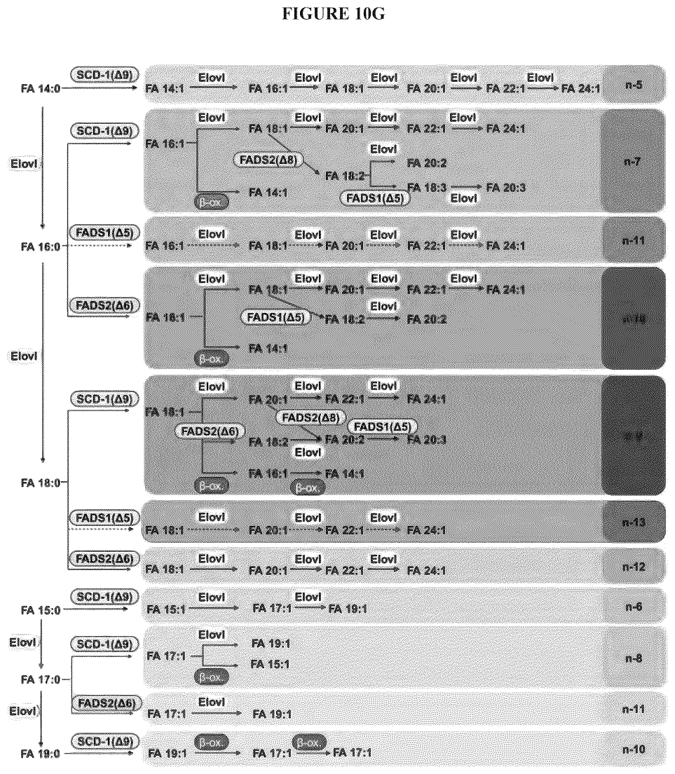

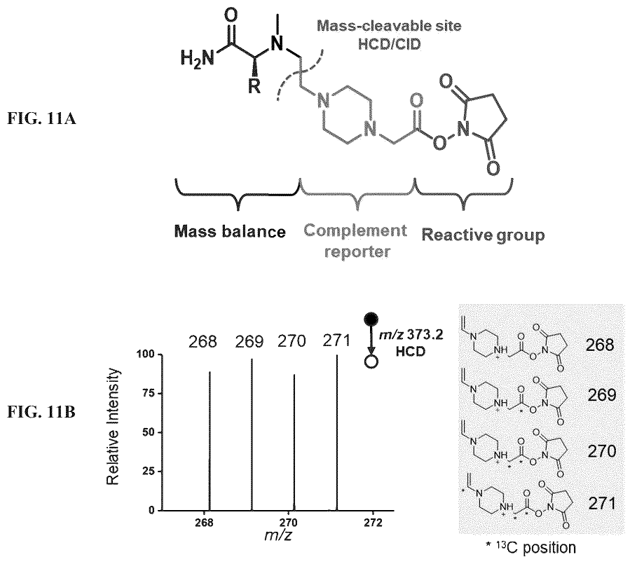

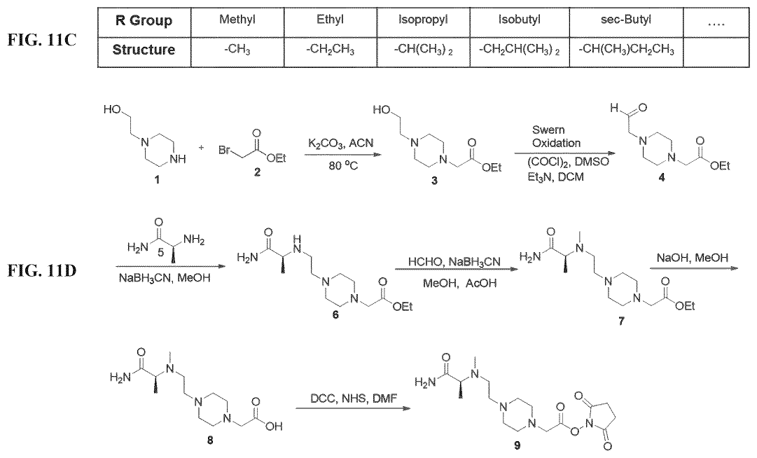

In total, 83 FA isomers were identified, and their concentration ratios (SCDi or SCDi+FADS2i vs. control) are shown in FIG. 9C. Hierarchical cluster analysis (HCA) shows good reproducibility within groups and clear discrimination between the two cell lines and treatments, while principal component analysis (PCA) of the C═C isomer profiles also distinguished cell types (P<0.0001) and treatment conditions (P=0.004) (FIG. 10A, FIG. 21A)—a contrast to sum-level profiles, which failed to do so (FIG. 10B). Notably, it was found that the differentiation of cell lines based upon PCA of C═C isomers is influenced strongly by the presence of FADS2 desaturated lipids as evidenced in FIG. 10C, FIG. 21B. In both cell lines, SCD-desaturated FAs such as FA 16:1 n-7 and FA 18:1 n-9 decreased after SCD inhibition (FIG. 9C, FIG. 10D). However, FADS2-derived FAs (e.g., FA 16:1 n-10, FA 18:1 n-10) increased exclusively in A 549 after SCD1 inhibition (FIG. 10E) and dropped significantly upon dual inhibition. In MDA-MB-231 none of these FAs show an increase. These results collectively suggest that the different proliferation responses between these two cells are due to the different usage of FADS2 alternative pathway.

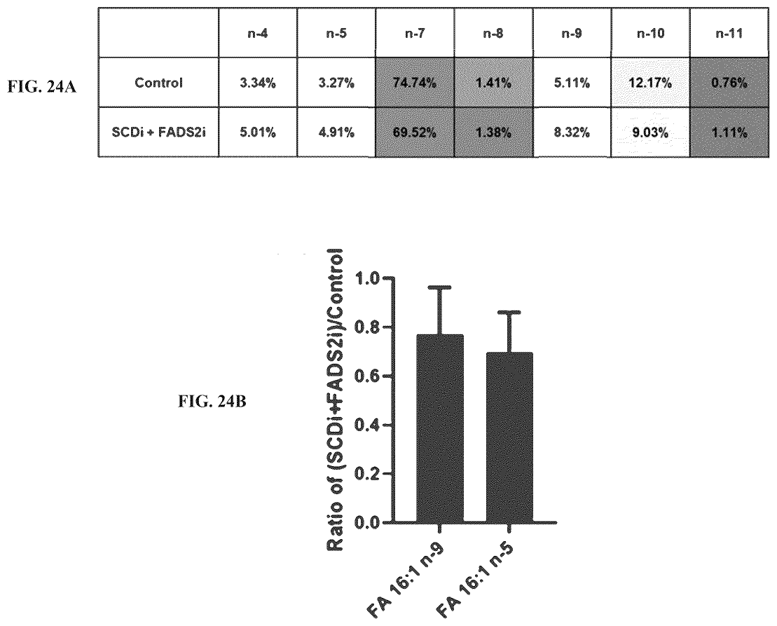

To compare the relative quantification method with a traditional composition-based approach, the composition of FA 16:1 isomer in A 549 cell after SCD inhibition, dual inhibition, and in controls was calculated and compared them for the relative quantification (FIG. 24A-FIG. 24B). For SCD inhibition alone, both methods yielded similar results. However, in the relative quantification between dual inhibition and control group, the composition-based method indicated an increase in FA 16:1 n-9 from 5.11% to 8.32% (1.63-fold) while the tagging approach shows the relative abundance of this isomer is decreased compared to control (ratio=0.77, FIG. 10D). Theoretically inhibiting SCD should lead to the decrease of FA 16:1 n-9. The reason that using composition information gives the opposite information is because the suppression of the n-10 isomer after dual inhibition shifting the relative composition toward the n-9, leading to an increase of its composition, as changes in composition are influenced by changes in other isomers. This demonstrates the advantage of the tagging approach as the results are not confounded by changes in other lipid isomers.

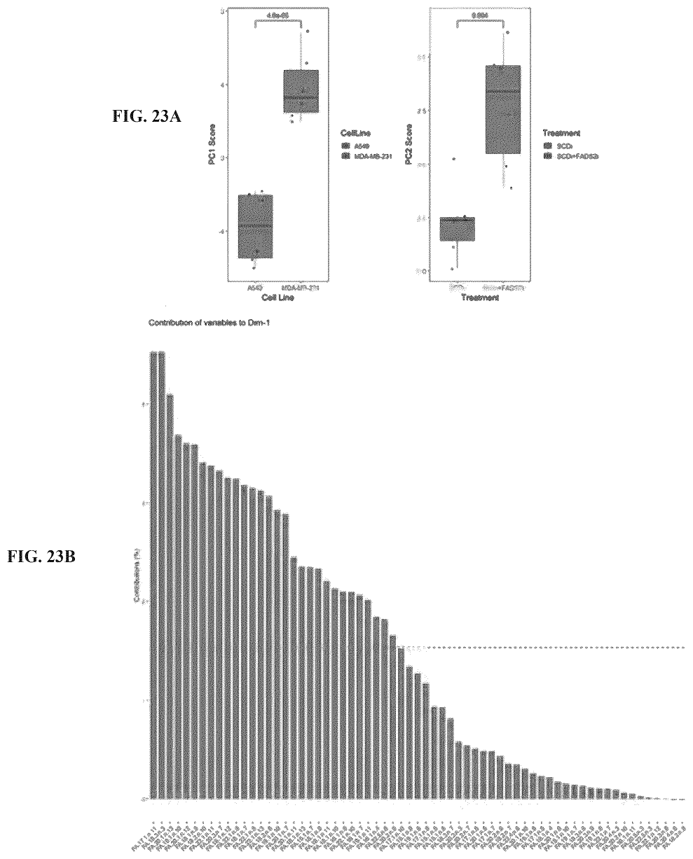

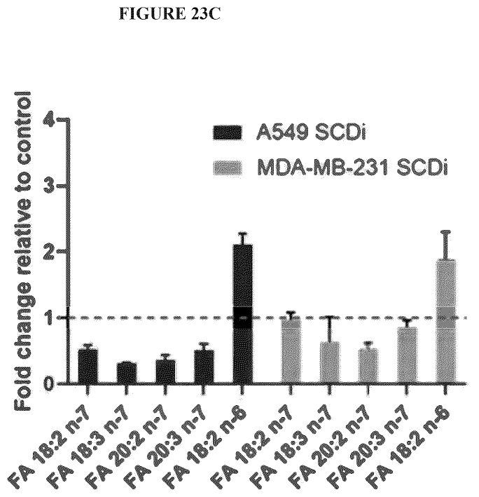

It was also observed that some FADS2-desaturated FAs (e.g., FA 22:1 n-10) did not increase in A 459; instead, other isomers such as n-11 and n-13 increase relative to control (FIG. 9C, FIG. 10F). Previous studies reported that FADS1 seems to be responsible for these uncanonical unsaturated FAs, and that inhibiting SCD can lead to an increase of n-11 and n-13 isomers. Therefore, it was speculated that inhibiting SCD may consolidate FADS1 activity in A 459, allowing it to compensate for the loss of unsaturated FAs by synthesizing FADS1-derived lipids rather than FADS2-derived lipids. Additionally, several de-novo synthesized PUFA, such as FA 18:3 n-7, were detected that can elongate to FA 20:3 n-7 in both A 549 and MDA-MB-231. This increased isomeric complexity of PUFA profiles likely involves SCD-mediated desaturation using MUFAs as substrates, resulting in an overall decrease in these PUFAs after SCD and FADS2 inhibition, whereas the dietary-derived FA 18:2 n-6 level increased (FIG. 23C). These PUFAs are remarkable not only for their biosynthetic origins and C═C modifications—achieved solely via cellular mechanisms—but also because they resemble dietary-derived, biologically active fatty acids such as dihomo-γ-linolenic acid (FA 20:3 n-6). This expanded isomeric complexity creates an array of unusual monounsaturated FAs, each reflecting specific enzyme substrate activities that can be visualized within a metabolic network (FIG. 10G). For example, FA 24:1 n-7 aligns with the canonical SCD desaturation of FA 16:0 to FA 16:1 n-7, followed by further chain elongation to produce a n-7 family that decreases upon SCD inhibition (FIG. 9C, Table 1). Conversely, the same FA 16:0 substrate may undergo FADS2 desaturation to yield FA 16:1 n-10, forming a family that extends to FA 24:1 n-10; these lipids are sensitive to FADS2 inhibition, showing significant decreases relative to the control (FIG. 9C, Table 2).

TABLE 1

| Lipid Classes | Type of Overlap | Example |
|---|---|---|
| PE O - LPE | PE O = LPE | [PE O − 26:1 + H] += [L PE 26:1 + H]+ |
| PE O LPC | PC O = LPC | [PC O − 26:1 + H] += [L PC 26:1 + H]+ |
| PC - PE | PC = PE + 3CH₂ | [PC 32:0 + H] += [PE 35:1 + H]+ |
| | | [PC 32:0 + Na] += [PE 35:0 + Na]+ |
| PC - PA | [PC + H} += [PA + 5CH₂ + DB + | [PC 32 + H] += [PA 37:1 + NH₄]+ |
| | NH₄]+ | |
| PE - PA | [PE + H} += [PA + C₂H₄ + DB + | [PE 34:1 + H] += [PA 36:2 + NH₄]+ |
| | NH₄]+ | |
| PS - PG | [PS + H] += [PG + 2DB + NH₄]+ | [PS 34:1 + H] += [PG 34:2 + NH₄]+ |
| SM - CerPE | SM = CerPE + 3CH₂ | [SM d34:1 + H] += [CerPE d37:1 + H]+ |

DB is a double bond

TABLE 2

| Lipid Classes | Type of Overlap | Δm/z | Example |
|---|---|---|---|
| Glycerolipids | [M] ≈ [M + CH₂ + O] | 0.036 | [PC 33:1 + H] +≈ [PC O − 34:1 + H]+ |
| Sphingolipids | [M] ≈ [M + CH₂ − DB − OH] | 0.036 | [SM t42:2] ≈ [SM d43:1] |
| PI - PS | [P] ≈ [PS + 6CH₂ + 5DB + | 0.002 | [PI 34:1 − H] −≈ [PX 40:6 − |
| | \hat{ }13C] | | H + \hat{ }13C]− |
| PC - SM | [PC + respective IS] ≈ [SM − | 0.065 | [PC 38:3 + ¹³C] ≈ [SM d42:2] |
| | OH₂ + 4CH₂ − DB] | | |
| DB containing | [M + Na] +≈ [M + 2CH₂ + 3DB + | 0.002 | [PC 34:1 + Na]\hat{ } +≈ [PC |
| classes | H]+ | | 36:4 + H]\hat{ }+ |
| PC - PS | [PC + H] +≈ [PS + DB + H]+ | 0.073 | [PC 32:0 + H] +≈ [PS 32:1 + H]+ |
| DG - CD | [CD X:Y + NH₄] +≈ [DG | 0.015 | [CD 18:1 + NH₄] +≈ [DG |
| | X + 30:Y + NH₄]+ | | 38:1 + NH₄]+ |

DB is a double bond.
O is an ether-linkage instead of ester-linkage.

### Example 12

### Materials and Methods of Examples 13-15

### Reagents and Materials

All lipid standards were purchased commercially. Ethyl oleate and fatty acids (FA) were purchased from Sigma-Aldrich (MO, USA), phosphatidylethanolamine (PE), and phosphatidylcholines (PC) were purchased from Avanti Polar Lipids (AL, USA). Cholesteryl ester (CE) and triacylglycerol (TAG) were purchased from Nu-Chek Prep (MN, USA). N-Boc-O-tosyl hydroxylamine (TsONHBoc) was purchased from Combi-Blocks (CA, USA). 1-(2-Hydroxyethyl)piperazine and Ethyl bromoacetate were purchased from Sigma-Aldrich (MO, USA). Ethyl bromoacetate (1-¹³C, 99%), Ethyl bromoacetate (1,2-¹³C₂, 99%), L-Alanine (1-¹³C, 99%), L-Alanine (2,3-¹³C₂, 99%) and Formaldehyde (¹³C, 99%) 20% w/w in H₂O were purchased from Cambridge Isotope Laboratories (MA, USA). Ammonium formate was purchased from Sigma-Aldrich (M O, USA) and formic acid was purchased from Fisher Scientific (NH, USA). HPLC grade acetonitrile, ethyl acetate, water, and isopropanol were purchased from Sigma-Aldrich (MO, USA). Hexafluoro-2-propanol (HFIP) was purchased from CHEM-IM PEX (IL, USA). TMT 6-plex isobaric label reagent set was purchased from Thermo Fisher Scientific (MA, USA). All solvents and chemicals were used without further purification.

### Synthesis of TBLT

### Ethyl 2-(4-(2-hydroxyethyl)piperazin-1-yl)acetate (3)

To a stirred suspension of 1-(2-hydroxyethyl)piperazine 1 (1 g, 7.68 mmol) and K₂CO₃ (1.6 g, 11.52 mmol) in CH₃CN (100 mL) was added ethyl bromoacetate (0.93 ml, 8.45 mmol) at 0° C. The reaction mixture was heated under reflux for 8 hr., diluted with EtOAc, and washed with water and brine. The extract dried over Na₂SO₄, concentrated to give the crude product, which was then purified by column chromatography over silica gel (dichloromethane/methanol=95:5) to give the ethyl 4-(2-hydroxyethyl)-1-piperazinylacetate 3 (1.56 g, 94%) as a yellow oil.

### Ethyl 2-(4-(2-oxoethyl)piperazin-1-yl)acetate (4)

To a solution of oxalyl chloride (1.2 mL, 13.88 mmol) in anhydrous CH₂Cl₂ (30 mL) at −78° C. DMSO (1.97 mL, 27.76 mol) was added over 5 min. After stirring at −78° C. for 30 min, a solution of the alcohol 9 (1.5 g, 6.94 mmol) in anhydrous CH₂Cl₂ (15 mL) was added. The reaction mixture was stirred at −78° C. for 1 hr., then triethylamine (6 mL, 41.64 mmol) was added, and the mixture was stirred at room temperature for 1 hr. The turbid mixture (monitored by TLC) was diluted with dichloromethane (30 mL) and washed with water (10 mL) and brine (10 mL). The organic layer was dried over anhydrous Na₂SO₄, filtered and concentrated in vacuo. The crude product was used without further purification.

### Ethyl(S)-2-(4-(2-((1-amino-1-oxopropan-2-yl)amino)ethyl)piperazin-1-yl)acetate (6)

To a stirred solution of compound 4 (1.5 g, 7 mmol) in methanol (30 mL), L-alaninamide hydrochloride (1.06 g, 8.4 mmol), NaBH₃CN (8.80 g, 14 mmol) and AcOH (1 mL) were added under N₂ atmosphere. The reaction mixture was stirred at room temperature overnight. The reaction mixture was concentrated under reduced pressure and then diluted with dichloromethane (30 mL) and water (7 mL). The mixture was basified to pH=9 with ammonium hydroxide. The aqueous phase was re-extracted with dichloromethane (2×15 mL). The organic layers were combined, washed with water (10 mL) and saturated brine (10 mL). The organic layer was dried over Na₂SO₄, filtered and concentrated under reduced pressure. The crude product was purified by column chromatography on silica gel, eluting with dichloromethane/methanol (9:1) to afford the compound 6 (891 mg, 45% over two steps) as a faint yellow oil.

### Ethyl(S)-2-(4-(2-((1-amino-1-oxopropan-2-yl)(methyl)amino)ethyl)piperazin-1-yl)acetate (7)

To a stirred solution of compound 6 (200 mg, 0.70 mmol) in methanol (4 mL), 20% aqueous formaldehyde (47 mg, 1.54 mmol), NaBH₃CN (109 mg, 1.75 mmol) and A cOH (0.36 mL) were added under N 2 atmosphere. The mixture was stirred at room temperature overnight. The reaction mixture was concentrated under reduced pressure and then diluted with dichloromethane (4 mL) and water (2 mL). The mixture was basified to pH=9 with ammonium hydroxide. The aqueous phase was re-extracted with ethyl acetate (3×4 mL). The organic layers were combined, dried over Na₂SO₄, filtered and concentrated. The crude product was purified by column chromatography on silica gel, eluting with dichloromethane/methanol (9:1) to afford the compound 7 (188 mg, 90%) as a yellow oil.

### (S)-2-(4-(2-((1-amino-1-oxopropan-2-yl)(methyl)amino)ethyl)piperazin-1-yl)acetic Acid (8)

To a stirred solution of compound 7 (150 mg, 0.5 mmol) in ethanol (25 mL) a solution of NaOH in H₂O (1 N, 2.5 mL) was added at room temperature. The reaction mixture was stirred at room temperature overnight. The mixture was concentrated under reduced pressure and diluted with dichloromethane (7.5 mL) and water (1.5 mL). The mixture was acidified to pH=6 with 1 N HCl aqueous solution. The aqueous phase was re-extracted with dichloromethane (2×7.5 mL). The organic layers were combined, dried over Na₂SO₄, filtered and concentrated under reduced pressure to afford the compound 8 (127 mg) as a white solid.

### 2,5-dioxopyrrolidin-1-yl(S)-2-(4-(2-((1-amino-1-oxopropan-2-yl)(methyl)amino)ethyl)piperazin-1-yl)acetate (9)

Compound 8 (20 mg, 0.07 mmol) and dicyclohexylcarbodiimide (DCC) (18 mg, 0.09 mmol) were dissolved in N, N-dimethylformamide (0.1 ml) and stirred at room temperature for 10 min. After N-hydroxy succinimide (10 mg, 0.09 mmol) was dissolved N, N-dimethylformamide (0.1 mL), it was added dropwise to the reaction solution. After 16 hours, the reaction mass was filtered on celite and washed with DM F (0.2 mL), filtrate was concentrated under reduced pressure. Dichloromethane was added to the residue and stirred for 15 min at room temperature to get precipitation, filtered to afford compound 9 (13.5 mg, 50% over two steps) as a white solid.

### Lipid Aziridination

Lipid standards were dissolved in hexafluoroisopropanol (HFIP) and mixed with 5 mM TsONHBoc. The reaction mixture was stirred at room temperature overnight

### Isobaric Tag Labeling of Aziridine Lipids

Isobaric TBLTs were dissolved in DM F to prepare as 35 mM stock solution and stored in −80° C. Lipids after aziridination were evaporated to remove HFIP solvent, then redissolve in ethanol solvent. The aziridine lipids were labeled with isobaric tag (20-100 equivalents) in DM F/EtOH solvent. The reaction mixture was shaken at room temperature for 10 hours.

### MS and HPLC Setting

Orbitrap Fusion Tribrid mass spectrometer (Thermo Fisher Scientific) coupled with vanquish UHPLC system (Thermo Fisher Scientific) was used for data acquisition.

### Example 13

### Design of Tandem Balance Loss Tag (TBLT)

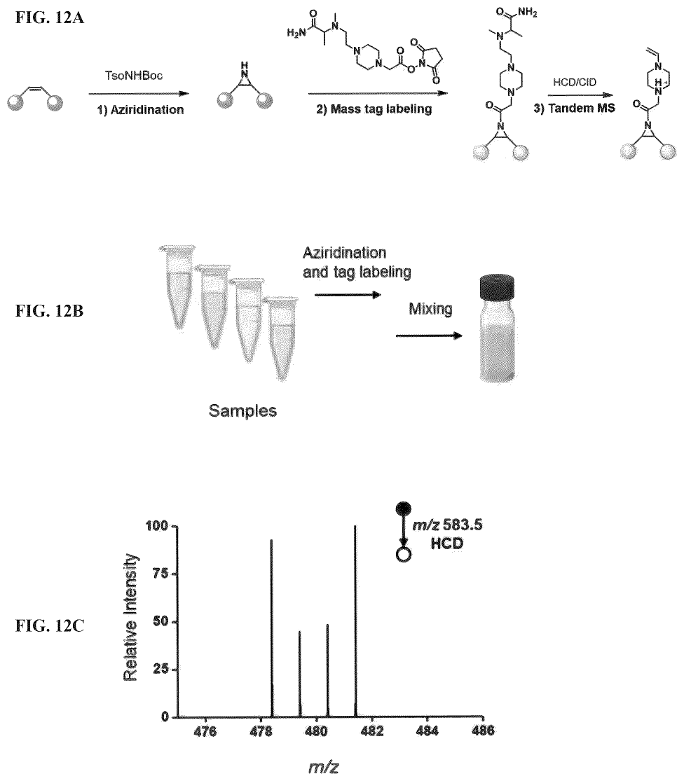

TBLT comprises three functional components: a mass balance group, a complementary reporter group, and an amine-reactive group (FIG. 11A). The amine reactive group is N-Hydroxysuccinimide (NHS), which is widely applied in biochemistry. The complementary reporter group is piperazine and the mass balance part is N-Methyl amino acid amide. A new type of mass cleavage bond was developed to connect mass balance and reporter parts. Upon higher-energy collision dissociation (HCD) or collision-induced dissociation (CID), the carbon-carbon bond linking the piperazine ring to the mass balance breaks efficiently, releasing the balance group. The complementary reporter group will remain covalently attached to the labeled molecule, generating diagnostic reporter ions suitable for quantification. These reporter ions exhibit high sensitivity due to both the efficient cleavage of the designed bond and the high ionization efficiency of the piperazine moiety.

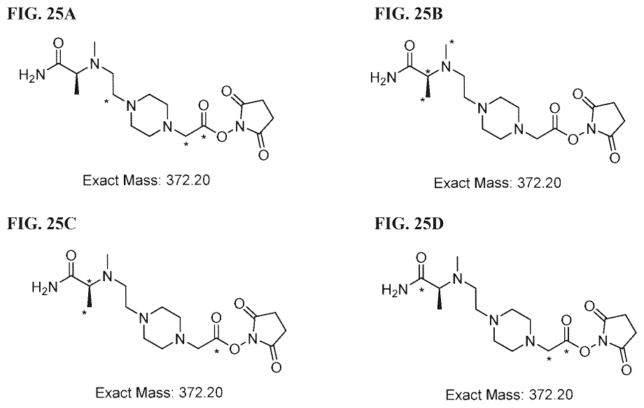

The tag design allows the introduction of up to nine heavy stable isotopes (two ¹⁵N and seven ¹³C) into the reporter group, significantly enhancing multiplexing potential. By filling out all combinations of positions in the reporter group with heavy atoms and counting the subtle mass difference between ¹⁵N and ¹³C, a theoretical maximum of 18-plex tagging can be achieved. Alternative R groups in the mass balance part—illustrated in FIG. 11C—can be selected based on the number of available isotope incorporation sites needed. In this study, a proof-of-concept is demonstrated by synthesizing a 4-plex isobaric tag version using methyl as the mass balance part. The structures of 4 isobaric tags were shown in FIG. 25A-FIG. 25D and corresponding mass reporter ions obtained upon HCD were presented in FIG. 11B.

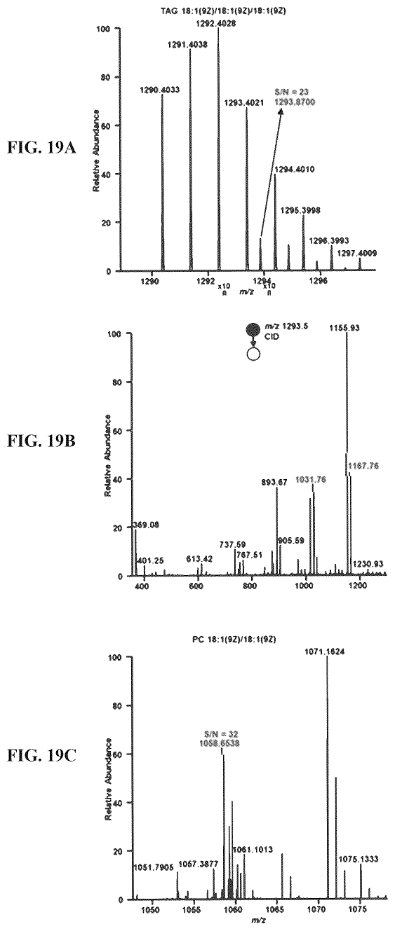

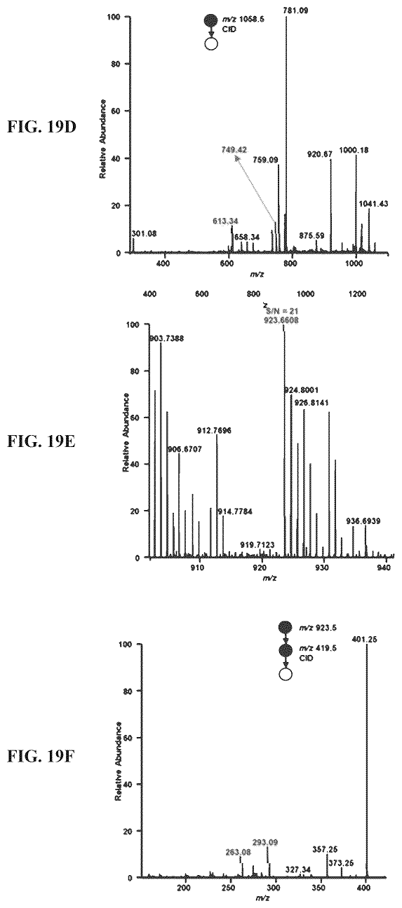

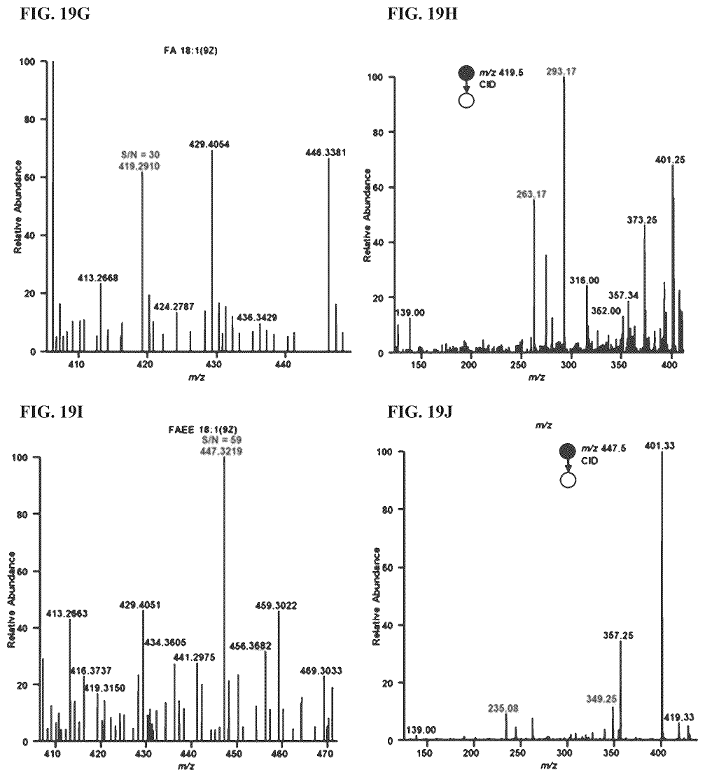

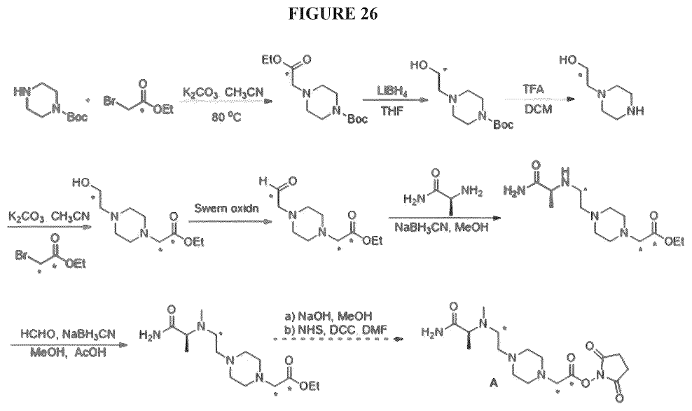

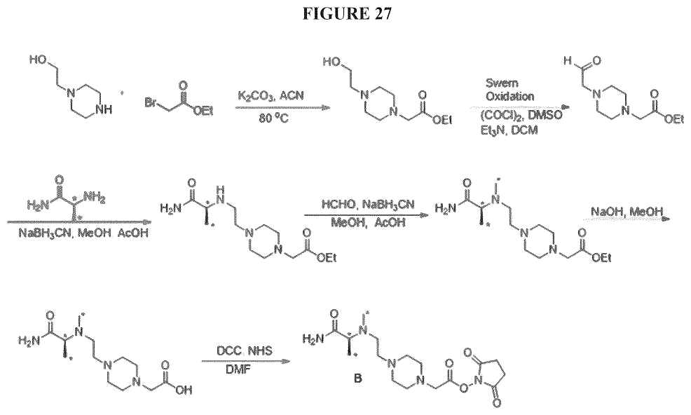

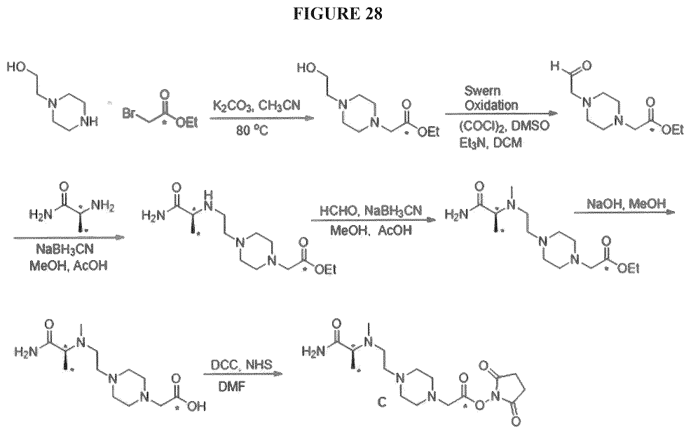

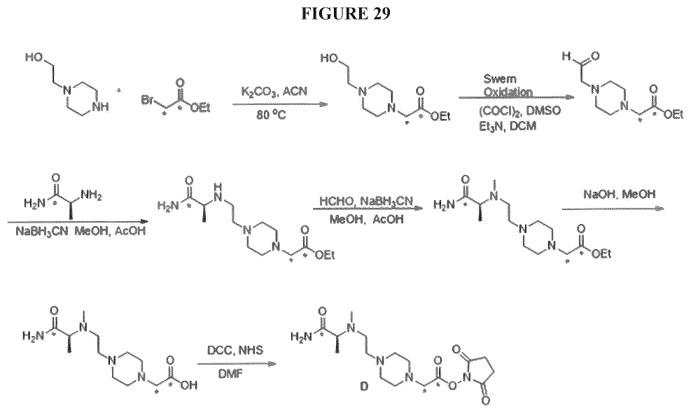

The synthetic route for non-isotopic tag is present in FIG. 11D. For synthesizing isobaric tags, different isotope-labeled reagents are selected and incorporated using this synthetic pathway. The detailed synthetic schemes for each isobaric TBLT are shown in FIG. 16-FIG. 29. A key advantage of the tag design is that balance groups can be derived directly from amino acids. For instance, using methyl as the R group allows the balance unit to be synthesized from alanine, which is available in multiple commercially labeled isotopic forms. This strategy reduces synthetic complexity, minimizes the number of required steps, and improves overall efficiency.

### Example 14

### TBLT Labeling of Lipid Aziridines for Relative Quantification at the Sum Level

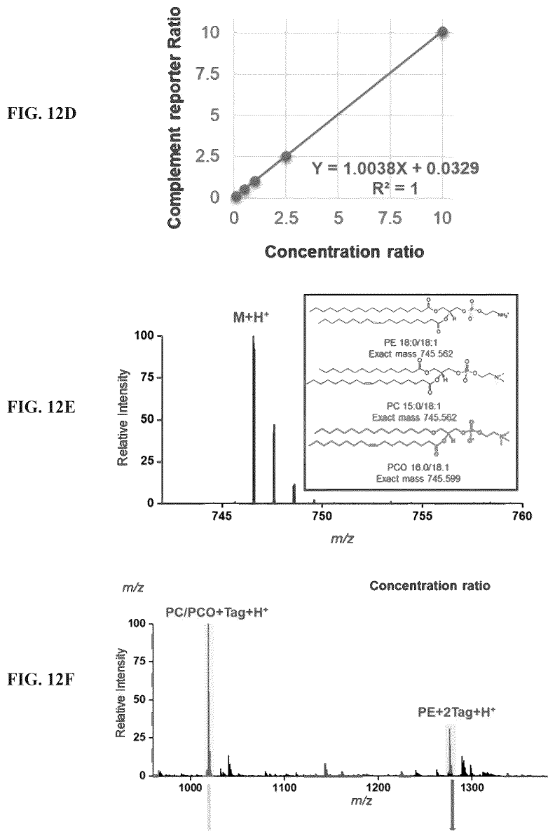

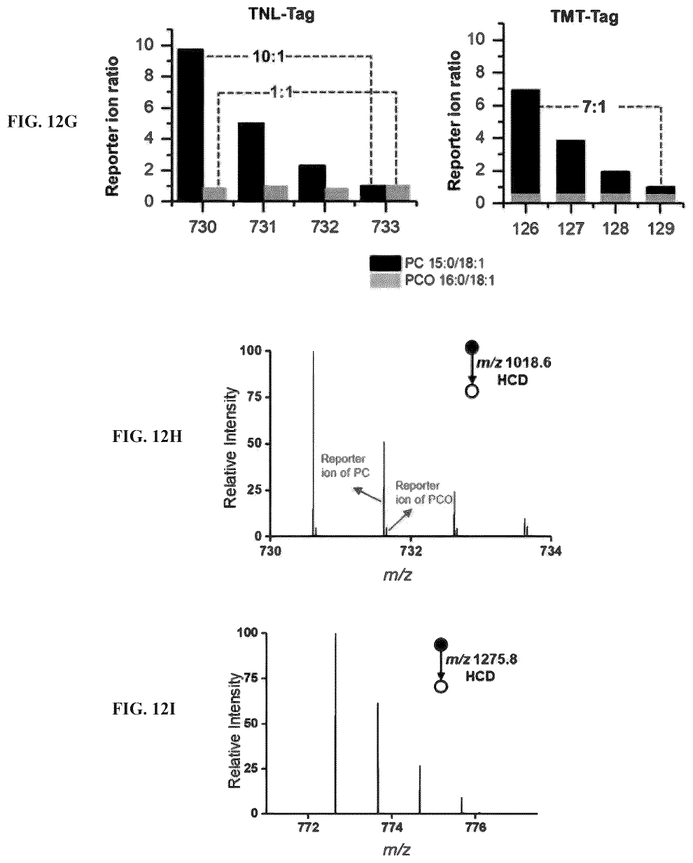

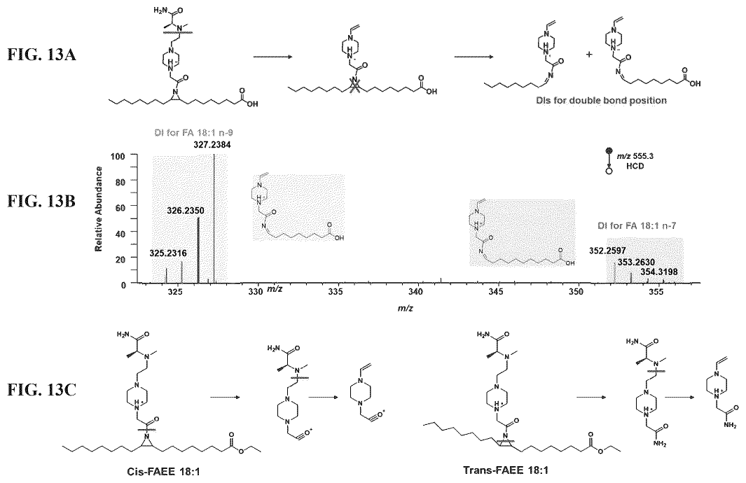

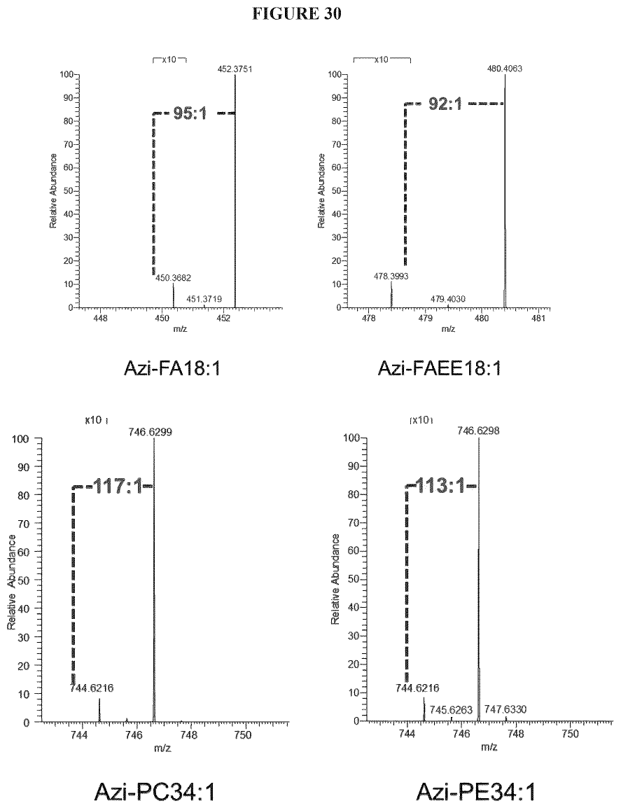

The mechanism of chemical derivatization and labeling strategy was illustrated in FIG. 12A, where lipid C═C bond is converted to aziridines via aziridination reaction, followed by TBLT labeling on the nitrogen atoms via NHS chemistry. Upon tandem MS, the mass balance groups are efficiently released, while the complementary reporter ions remain attached to the lipid backbone, which could be used for relative quantification. To assess the quantitative abilities of isobaric TBLT ethyl oleate was selected for derivatization and analysis. Four different samples with ethyl oleate at the concentration ratio of 2:1:1:2 were prepared, followed by the aziridination and tag labeling steps (FIG. 12B). Then the four samples were mixed to remove the matrix effect and subjected to MS detection followed by HCD. The resulting MS/MS spectrum (FIG. 12C) revealed four distinct complementary reporter ions at m/z 478, 479, 480, and 481, representing lipids originating from different samples. The measured intensity ratios of these reporter ions directly reflected the lipid concentration ratios as 2.0:0.9:1:2.1, closely matching the theoretical values. To further validate the quantification capability across a broad range of lipid concentrations, ethyl oleate aziridine was prepared and tagged at the concentrations of 1 μM, 5 μM, 10 μM, 25 μM, 100 μM, and the concentration ratios from different samples were kept at 0.1, 0.5, 1, 2.5 and 10. The correlations between measured ratios of mass reporters and expected concentration ratios were displayed in FIG. 12D. Linear relationships were presented with R² equal to 1 and a slope of 1.004. Higher ratios (up to 1:100) were also tested using various lipids such as ethyl oleate, oleic acid, PC 34:1, and PE 34:1, with consistent quantification results (FIG. 30). These results highlight TBLT's great quantification performance, demonstrating its accuracy, sensitivity, broad dynamic range, and versatility across diverse lipid classes.

Another significant advantage of TBLT labeling is its ability to minimize ratio distortion caused by co-isolation of interfering precursor ions, a common issue encountered with conventional mass tags. Given the structural diversity of lipids, many species share same or very similar molecular compositions (Table 1 and Table 2). With traditional mass tag labeling, simultaneous fragmentation of co-isolated interfering precursors can produce indistinguishable reporter ions, resulting in distorted quantification. In contrast, the TBLT approach retains the precursor mass information within the lipid-coupled reporter ions, enabling differentiation between target and interfering lipids, thereby achieving quantification. To demonstrate this capability, three isomeric or isobaric lipid species were selected. Specifically, PC 33:1, PE 36:1, and PCO 34:1 exhibit same or similar exact mass, as shown in FIG. 12E. Four samples were prepared with the ratio of PC 33:1, PCO 34:1 and PE 36:1 at 10:5:2.5:1, 1:1:1:1 and 10:5:2.5:1 respectively. After TBLT labeling, mass peaks of labeled PC/PCO and PE could be differentiated due to different number of labels tags, arising from structural differences: PC 33:1 and PCO 34:1 each contain a single amine from double bond aziridination, whereas PE 36:1 contains two amines—one from the double bond aziridination and one from its head group (FIG. 12F). Then, the precursors were isolated at m/z 1018.6 and 1275.8 and perform tandem MS respectively. FIG. 12H illustrates that the slight mass differences between the reporter ions of PC 33:1 and PCO 34:1 were clearly resolved, enabling separate quantification. FIG. 12I also shows good agreement between the reporter ion intensities and the known concentration ratios of PE 36:1. For comparison, when using commercial TMT labeling, labeled PC and PCO precursors are co-isolated during MS/MS analysis due to their closely similar masses, releasing same reporter ions. This makes it impossible to distinguish their individual contributions, causing ratio distortion. Specifically, the observed intensity ratio (7:1) deviated from the true concentration ratio (10:1 and 1:1), as shown in FIG. 12G.

### Example 15

### Characterization and Quantification of Lipids at Multiple Isomer Levels

TBLT labeled lipids can produce a set of complementary reporter ions: (1) mass reporter coupled to the lipid backbone, formed by only loss of mass balance group and (2) mass reporter coupled to structural diagnostics ions (DIs), formed during the further fragmentation upon HCD. These DI-coupled reporter ions facilitate comprehensive structural identification and quantification at multiple isomeric levels, including double bond positional isomers, geometric (cis/trans) configurations, and sn-positional isomers.

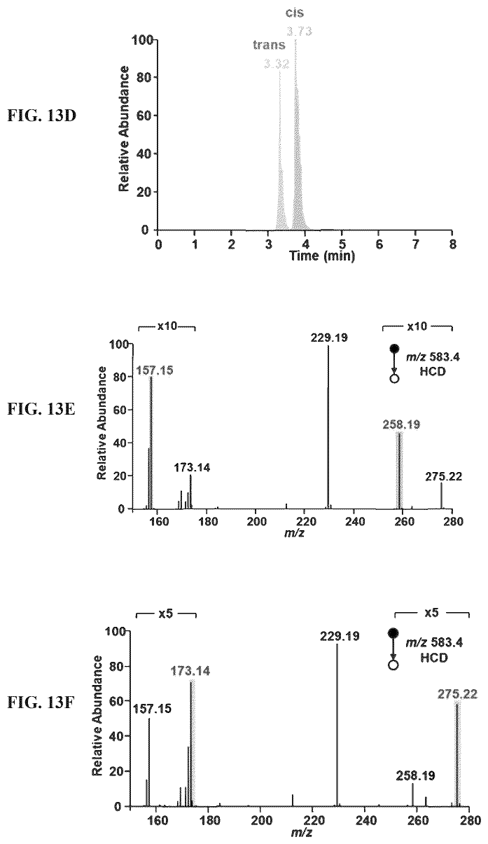

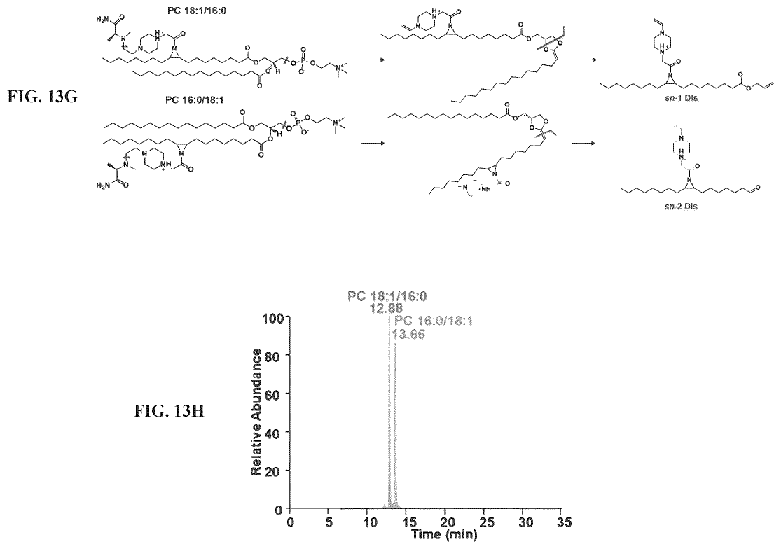

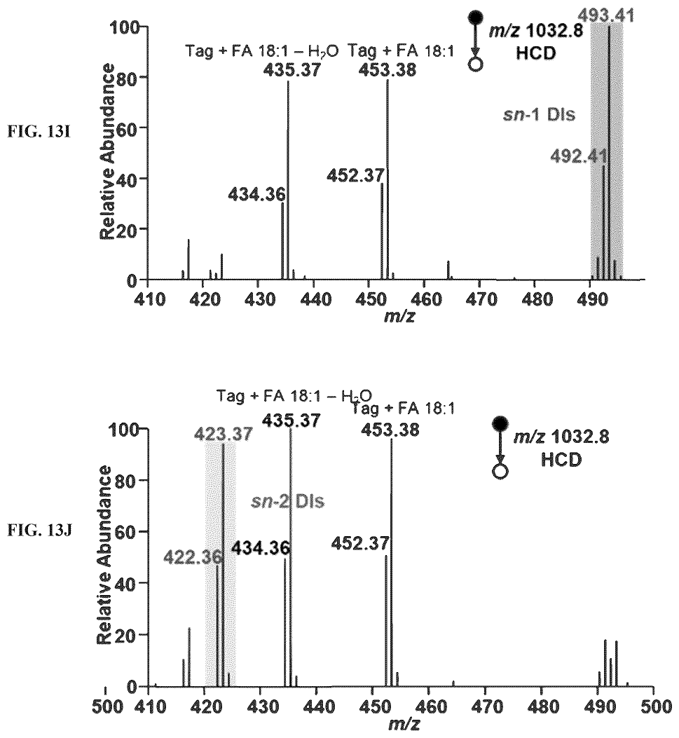

Double bond positions can be pointed by the DIs generated from the cleavage of the aziridine ring, as illustrated in FIG. 13A. Importantly, the pair of DIs retains the mass reporter portion, enabling simultaneous identification and quantification of different double bond positional isomers. To validate the capability of TBLT for quantifying double bond positional isomers, two isomers, FA 18:1(n-7) and FA 18:1(n-9) were selected for derivatization and analysis. Four samples containing these isomers at varying ratios (FA 18:1(n-7): 1:2:5:10; FA 18:1(n-9): 10:5:2:1) were prepared at a constant total concentration of 100 μM. After derivatization and tag labeling, the samples were mixed and subjected to MS detection. The peak of tag-labeled FA 18:1 at m/z 555.3 were selected as the precursor ions for fragmentation, generating distinct DIs coupled with reporter groups for n-7 and n-9 isomers upon HCD, enabling simultaneous identification and quantification of each positional isomer within the mixture (FIG. 13B).

Different fragmentation behavior of cis/trans isomers were also observed after TBLT labeling. As illustrated in FIG. 13C, the cis fatty acid ethyl ester 18:1 (cis-FAEE 18:1) and trans fatty acid ethyl ester 18:1 (trans-FAEE 18:1) shows different cleavage preferences of detaching tag during HCD. Specifically, cis-FAEE 18:1 preferred to undergo carbon-nitrogen bond cleavage at the amine group forming detached carbonyl tag, while trans-FAEE favored cleavage at the aziridine ring forming detached amine tag. This cleavage difference was evident in their tandem MS spectra in FIG. 13E and FIG. 13F. For cis-FAEE, detached carbonyl tag ion at m/z 258 and its corresponding balance-loss ions exhibited higher intensities compared to detached amine tag ion at m/z 275. Conversely, trans-FAEE showed an opposite intensity trend. Furthermore, after TBLT labeling, these geometric isomers were chromatographically separable by HPLC. Thus, the cis/trans isomers can be reliably identified based on the fragment pattern after separation and be quantified using the mass reporter ions in the mean while.

Lastly, TBLT labeling helps effectively distinguishes sn-positional isomers in glycerophospholipids. The sn-isomers, PC 18:1/16:0 and PC 16:0/18:1, were investigated as an example in this study. As shown in FIG. 13G, dioxolane-induced cross-ring cleavage generated different fragments ions for sn-positional isomers. These diagnostic fragments of acyl chains also carried mass reporter parts, which could be used for quantification of sn-positional isomer. To demonstrate quantitative accuracy, two samples of PC 18:1/16:0 were labeled with different isobaric TBLT at a 2:1 concentration ratio. The tandem MS spectrum of two samples mixture accurately reflected this ratio through the intensity's ratio of sn-1 positional DIs at m/z 492 and 493 (FIG. 3I). Similar experiment performed with PC 16:0/18:1 also yielded an intensity ratio of sn-2 positional DIs at m/z 422 and 423 accurately reflecting the 1:2 concentration ratio (FIG. 13I). Additionally, HPLC separation of TBLT-labeled sn-isomers further facilitated precise identification and quantification based on these DIs.

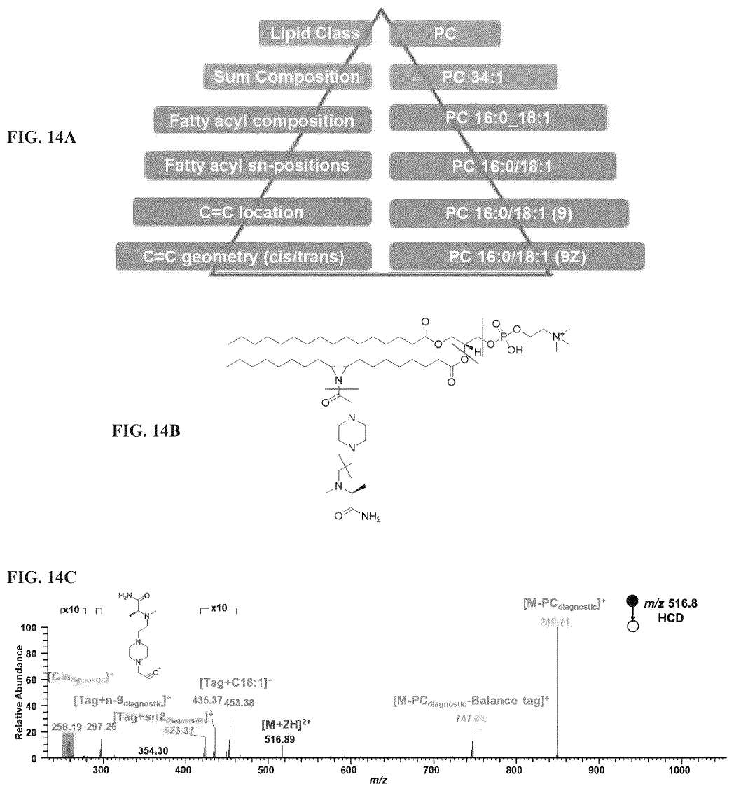

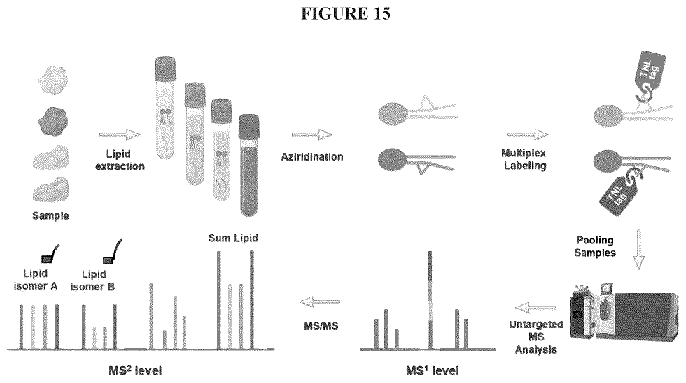

Overall, the unique fragmentation pattern of TBLT-labeled lipids generates comprehensive sets of diagnostic ions, enabling simultaneous structural identification and precise quantification at multiple lipid isomer levels. By integrating these DIs, an in-depth structural characterization of lipids encompassing subclass, fatty acyl chain length, double bond positions, cis/trans configurations, and sn-positional isomerism is achievable within a single tandem MS analysis, as exemplified with PC 16:0/18:1(9Z) in FIG. 14A-FIG. 14C.

The tandem balance loss tag (TBLT) represents a significant innovation in lipidomics, offering high accuracy and depth in structural characterization and quantification of lipids at multiple isomeric levels. With its unique fragmentation strategy, TBLT successfully addresses critical limitations inherent to conventional isobaric tags used for lipids, including ratio distortion and inadequate isomer resolution. Demonstrated across various lipid classes, TBLT showcases outstanding linearity, sensitivity, and specificity, firmly establishing its applicability for complex lipidomic studies.

### Example 16

Aziridination-Based ¹³C Mass Tags (Azi-¹³CMT) Kit Protocol

After lipid standards or lipid extracts from biological samples are collected, lipid can be aziridinated with Azi-¹³CMT kit. In the instant example, lipid aziridines are analyzed by a high-performance liquid chromatography (HPLC) coupled with a mass spectrometer (MS) to locate lipid C═C bond positions and to provide quantification of those isomers.

The Azi-¹³CMT kit of the instant example includes four reagents (4-nitroaniline, [¹³C₆]-4-nitroaniline, iodosobenzene (PhIO), and Rh₂(esp)₂ catalyst) and a solvent (hexafluoroisopropanol, HFIP). To avoid contamination of MS samples, gloves can be worn and ultrapure MS-grade reagents can be used.

The PhIO Reagent can be moisture sensitive. Thus, to avoid moisture condensation onto the product, the vial can be equilibrated to room temperature before opening. 4-Nitroaniline and [¹³C₆]-4-nitroaniline can be dissolved in HFIP and can also be stored at −20° C. for about one month. PhIO should be stored at −20° C. and freshly prepared before experimentation. The Rh₂(esp)₂ catalyst should be stored at room temperature and freshly prepared before experimentation. Finally, the 4-nitroaniline reagent can be used to optimize methods before analysis of samples with the [¹³C₆]-4-nitroaniline.

Additional materials to be used with the kit of the instant example can include microcentrifuge tubes, 1 mL Glass vials, dichloromethane (DCM) (e.g., LC-MS grade), and a reverse phase C30 column.

The reagent 4-nitroaniline solution can be prepared in solvent HFIP at a concentration of 8.28 mg/mL. Reagent [¹³C₆]-4-nitroaniline solution can be prepared in solvent HFIP at a concentration of 8.65 mg/mL. Reagent PhIO solution can be prepared in solvent HFIP at a concentration of 0.825 mg/mL. Reagent Rh₂(esp)₂ solution can be prepared in solvent HFIP/DCM (4/1, v/v) at a concentration of 3.79 mg/mL. Sonic the solution for 10 seconds before use.

The protocol below for the instant example provides an aziridination reaction of lipid extracts from two biological samples using the Azi-¹³ CM T kit and for the relative quantification. Approximately 10 to 250 μg of lipid extract is used for each labeling reaction according to the following procedure.

1. Add 50 μL DCM to each vial with the sample.

2. Add 100 μL 3.79 mg/mL Rh₂(esp)₂ to each sample.

3. Add 50 μL 8.28 mg/mL 4-nitroaniline to one sample and 8.65 mg/mL [¹³C₆]-4-nitroaniline to another sample.

4. Put the two vials on a stir plate and begin to stir the two reaction mixtures.

5. Add 800 μL 0.825 mg/mL PhIO solution to each reaction mixture while stirring.

6. Continue stirring at room temperature for 20 h.

7. After 20 h of reaction, mix the two samples in a 1/1 volume ratio and detect the mixed samples with HPLC-MS.

### Example 17

### Tandem Balance Loss Tag (TBLT) Mass Tagging Kit Protocol

In the instant example, lipids extracted and isolated from cells or tissues can be aziridinated overnight and then dried. Samples can be labeled with TBLT reagents, respectively, and then mixed for detection. Labeled samples can be analyzed by high-resolution LC-MS/MS before data analysis to identify lipid isomers and quantify reporter ions relative abundance.

The kit of the instant example includes a total of 4 reagents: two reagents for lipid aziridination (N-Boc-O-tosyl hydroxylamine, TsONHBoc, and hexafluoroisopropanol, HFIP) and two reagents for tag labeling (TBLT-4plex reagent, 1M triethyl ammonium bicarbonate buffer). The TBLT Reagents are moisture sensitive. To avoid moisture condensation onto the product, the vial should be equilibrated to room temperature before opening.

Anhydrous dimethylformamide can be used as a solvent to dissolve TBLT reagents. Stock solutions are stable for one week when stored at −20° C. For long-term storage, unused reagent can be stored at −80° C. or remove all solvent by drying and store with desiccant at −20° C. A voiding buffers and additives containing amine during lipid extraction is suggested. For optimal results, use 10-50 μg of lipids for each labeling reaction. To avoid contamination of MS samples, gloves can be worn and ultrapure MS-grade reagents can be used. The TBLTzero Label Reagent can be used to optimize methods before multiplexed analysis of samples with the TBLT-4plex Set.

Additional materials in the kit can include anhydrous dimethylformamide, anhydrous ethanol, lipid extraction solvents (e.g., Folch method using chloroform, methanol, water), ammonium bicarbonate or hydroxylamine for quenching the labeling reaction, a C18 reversed-phase column, and a high-resolution Mass Spectrometer with liquid chromatography (LC) system

For material preparation of the 1M ammonium bicarbonate buffer 100 μL water is added to dissolve 7.9 mg Ammonium bicarbonate. For preparation of 5 mM TsONHBoc, 3 mL HFIP is added to 4.3 mg TsONHBoc.

The protocol below for the instant example provides a procedure for preparing and labeling lipids with the TBLT Isobaric Mass Tags.

First, for lipid aziridination:

1. Prepare 10-50 μg extracted lipid from cells or tissues and transfer into a vial.

2. Add 200 μL 5 mM TsONHBoc into the vial.

3. Stir the reaction mixture at room temperature overnight on a stir plate.

4. Dry the reaction using rotary evaporation.

5. Redissolve the aziridinated lipids in 50 μL ethanol

Second, for lipid labeling:

1. Immediately before use, equilibrate the TBLT reagents to room temperature. Add 50 μL of anhydrous dimethylformamide to each vial containing 1 mg TBLT.

2. Add 50 μL TBLT reagent to each 50 μL sample (10-50 μg) and add 10 μL 1M TEAB into the reaction system.

3. Incubate the reaction for 10 hours at room temperature.

4. Add 5 μL 1M ammonium bicarbonate to the sample and incubate for 15 minutes to quench the reaction.

5. Combine samples in equal amounts in a new microcentrifuge tube and store at −80° C. before MS detection.

1. A kit comprising i) a plurality of reagents, wherein the reagents are selected from the group consisting of 4-nitroaniline, [¹³C₆]-4-nitroaniline, iodosobenzene (PhIO), Rh₂(esp)₂ catalyst, and any combination thereof and ii) a solvent.

2. The kit of claim 1, wherein the solvent is hexafluoroisopropanol (HFIP).

3. The kit of claim 1, further comprising instructions for use.

4. A kit comprising i) a plurality of reagents, wherein the reagents are selected from the group consisting of N-Boc-O-tosyl hydroxylamine, TsONHBoc TBLT-4plex reagent, 1M triethyl ammonium bicarbonate, and any combination thereof, and ii) a solvent.

5. The kit of claim 4, wherein the solvent is hexafluoroisopropanol (HFIP).

6. The kit of claim 4, further comprising instructions for use.

7. A method of identifying one or more lipid isomers in a sample, said method comprising contacting the sample with one or more isotopic labels to identify the one or more lipid isomers.

8. The method of claim 7, wherein the method comprises quantification of the one or more lipid isomers.

9. The method of claim 7, wherein the method comprises identification of one or more double bond positions of the one or more lipid isomers.

10. The method of claim 7, wherein the method comprises determination of a molar ratio of the one or more lipid isomers.

11. The method of claim 7, wherein the one or more lipid isomers are selected from the group consisting of fatty acids (FA), glycerophospholipids (GPL), unsaturated fatty acid derivatives, cholesteryl ester (CE), triacylglycerides (TAG), yeast polar extracts, and any combination thereof.

12. The method of claim 7, wherein the method further comprises administering collision-induced dissociation (CID) to the sample.

13. The method of claim 7, wherein the one or more isotopic labels comprises an aziridination-based labeling.

14. The method of claim 7, wherein the one or more isotopic labels comprises a deuterium-based labeling.

15. The method of claim 7, wherein the one or more isotopic labels comprises a ¹³C-based labeling.

16. The method of claim 7, wherein the one or more isotopic labels comprises a 2-aminopyridine (2-AP)-based labeling.

17. The method of claim 7, wherein the one or more isotopic labels comprises a 4-nitroaniline-based labeling.

18. The method of claim 7, wherein the one or more isotopic labels comprises an N-aryl-based labeling.

19. The method of claim 7, wherein the one or more isotopic labels comprises a 4-nitroaniline-based N-aryl aziridination labeling.

20. The method of claim 7, wherein the one or more isotopic labels comprises an aziridination-based and a tandem balance loss tag (TBLT) labeling.
